(19) United States
(12) Patent Application Publication
(10) Pub. No.: US 2019/0336453 A1
(43) Pub. Date: Nov. 7, 2019

(54) ORAL DISPERSIBLE FILM COMPOSITION

(71) Applicant: BonAyu Lifesciences Private Limited, Bengaluru District (IN)

(72) Inventors: Vishal Kataria, Bangalore (IN); Kailas Kalicharan Moravkar, Mumbai (IN); Wwikruti Sen, Bangalore (IN); Geeta Umesh Yadav, Mumbai (IN); Purnima Dhanraj Amin, Mumbai (IN)

(21) Appl. No.: 16/475,644

(22) PCT Filed: Jan. 4, 2018
(86) PCT No.: PCT/IN2018/050007

§ 371 Date: Jul. 2, 2019

(30) Foreign Application Priority Data
Jan. 4, 2017 (IN) 201741000424

(51) Int. Cl. A61K 9/70; A61K 47/38; A61K 47/10; A61K 9/00; A61K 8/73; A61K 8/02

(52) U.S. Cl. CPC A61K 9/7007; A61K 47/38; A61K 47/10; A61K 45/06; A61K 8/731; A61K 8/732; A61K 8/0216; A61K 9/006

(57) ABSTRACT

Pharmaceutical and nutraceutical composition in the form of oral dispersible films (ODFs) using twin-screw hot melt extrusion was described. In the present disclosure, there is provided an oral dispersible film composition comprising: (a) maltodextrin; and (b) hydroxypropyl cellulose, wherein the weight ratio of maltodextrin to hydroxypropyl cellulose is in the range of 1:1-3:1. The prepared films of pharmaceutical and nutraceutical composition are uniform in film thickness, have excellent physical attributes and can be directly packed after cutting.

### FIELD OF INVENTION

The present disclosure generally relates to the field of polymers and particularly to water soluble polymers. The present disclosure in particular relates to the field of pharmaceutical and cosmeceutical polymeric compositions. The present disclosure also describes the process of preparation of the oral dispersible film composition using twin-screw hot melt extrusion process.

### BACKGROUND OF THE INVENTION

Over the past few decades, rapid release dosage forms that release pharmaceuticals into the oral cavity are being investigated. Among all the fast release dosage forms, oral dispersible films (ODF) are among the most successful due to the ease of formulation. The ODFs are popular among a majority of patients because of their easily dissolvable, convenient and user friendly nature of the dosage form.

Typically an ODF is an ultrathin strip, which is similar to postage stamp in shape and size, with/without actives and mostly water soluble excipients like film forming polymers and plasticizers. The ODFs have larger surface area that leads to their rapid disintegration in oral cavity. ODFs are flexible enough with adequate ease of transport and handling. ODFs provide ease of swallowing and patient can take it even without water. Therefore, it is convenient for patients suffering from dysphagia, repeated emesis, motion sickness and mental disorders. With all the above mentioned assets, ODF is commercially a successful dosage form.

Initially, solvent casting method was used for manufacturing the ODFs. Solvent casting is a proven benchmark technology for ODF manufacturing because of high quality of product with better physical characteristics, ease in product development, process optimization, process validation and technology transfer to production scale. However, the technology had some drawbacks, namely, more number of unit operations involved and consumption of large quantity of solvents along with controlled limits of organic volatile impurities in final formulation. The use of organic solvents in the solvent casting method was a limiting parameter and a point of great concern for formulation scientists. The solvents used in the method were found to be flammable, as for example ethanol, and therefore required special safety equipment and procedures to be employed in order prevent fire and environmental hazards from vaporizing solvent. The solvent recovery also leads to extra cost. Moreover, the most hazardous drawback is the presence of solvent in traces in the final product which limits the compendial standards. Additionally, the manufacturing process is not environment friendly and has high carbon footprint.

The application of hot-melt extrusion in the pharmaceutical industry is consecutively increasing due to its proven innumerable advantages like provision for solvent-free continuous processing with fewer unit operations and better content uniformity. Hot melt extrusion is an industrially feasible process for continuous manufacturing. A single-screw extruder consists of one rotating screw positioned inside a stationary barrel at the most fundamental level. The single-screw extrusion system has three zones with lots of disadvantages. It does not acquire the mixing capability of a twin-screw extruder and therefore is not the preferred approach for the production of most pharmaceutical formulations in industry. Moreover, a twin-screw extruder offers much greater versatility (process manipulation and optimization) in accommodating a wider range of pharmaceutical and nutraceutical formulations making this setup much more constructive. Cilurzo et al. (Eur J Pharm Biopharm. 2008, 70(3), 895-900) explains the hot melt extrusion process of film using single screw extrusion, which has limited applications in industry.

Hot melt extrusion has more recently been applied to the health-care industry where it is used to manufacture medical devices and to mix active pharmaceutical ingredients (APIs) with polymers to enhance the API's bioavailability or prepare subcutaneous and intraocular implants and intra-vaginal rings.

The oral dispersible films prepared through the hot melt extrusion process would overcome the hazards of the solvent casting methods and would also impart desired attributes to the film composition.

### OBJECTIVE OF THE INVENTION

It has been already perceived that industrially scalable, continuous manufacturing of ODFs using water soluble polymers and their combinations has never been disclosed earlier. Additionally, the manufacturing of such formulation in one-step is added advantage. Also, the product can be directly packaged after manufacturing.

The principle object of the invention is to develop ODFs containing various actives by twin-screw hot melt extrusion as industrially scalable, continuous manufacturing process.

Another object of this invention is to develop various pharmaceutical and nutraceutical compositions of these ODF formulations like mouth freshener films, nutraceutical films and films containing vitamins and more.

Additional object of present invention is to reduce the number of unit operations and use of hazardous solvents in the formulation of films, thus making the process greener. This will increase the compendia compliance and thus consumer appeal.

### SUMMARY OF THE INVENTION

In an aspect of the present disclosure, there is provided an oral dispersible film composition comprising: (a) maltodextrin; and (b) hydroxypropyl cellulose, wherein the weight ratio of maltodextrin to hydroxypropyl cellulose is in the range of 1:1-3:1.

In an aspect of the present disclosure, there is provided a process for the preparation of oral dispersible film composition comprising (a) maltodextrin; and (b) hydroxypropyl cellulose, wherein the weight ratio of maltodextrin to hydroxypropyl cellulose is in the range of 1:1-3:1, said process comprising the steps of: (a) contacting maltodextrin, hydroxypropyl cellulose, plasticizer, at least one active ingredient, at least one saliva stimulating agent, and excipient to obtain a first mixture; and (b) extruding the first mixture to obtain the oral dispersible film composition.

In an aspect of the present disclosure, there is provided a system for the preparation of oral dispersible film composition comprising (a) maltodextrin; (b) hydroxypropyl cellulose and (c) plasticizer, wherein the weight ratio of maltodextrin to hydroxypropyl cellulose is in the range of 1:1-3:1, said system comprising: (a) a hopper zone for feeding the second mixture as described herein; (b) a conveying zone for transporting, processing and homogenizing the second mixture; (c) an orifice zone for shaping the homogenized second mixture to obtain a film of the second mixture; and (d) a downstream ancillary equipment zone for collecting the film of the oral dispersible film composition, wherein the oral dispersible film composition is in the form of a film having an average thickness in the range of 0.04-0.8 mm.

### BRIEF DESCRIPTION OF DRAWINGS

FIG. 1 illustrates cost comparison of various polymers in accordance with an implementation of the present disclosure.

FIG. 2 illustrates tensile strength evaluation of the oral dispersible film composition in accordance with an implementation of the present disclosure.

FIGS. 3a and 3b depict FE-SEM micrographs of film containing of the oral dispersible film composition obtained via single screw extrusion in accordance with an implementation of the present disclosure.

FIGS. 3c and 3d depicts Scanning Electron Microscopy (SEM) micrographs of film containing oral dispersible composition obtained via twin screw extrusion in accordance with an implementation of the present disclosure.

FIG. 4a represents 2D view of mouth freshener film prepared using single screw hot melt extrusion. FIG. 4b depicts phase diagram of oral dispersible film composition prepared using single screw hot melt extrusion. FIG. 4c depicts 3D view of oral dispersible film prepared using twin screw hot melt extrusion in accordance with an implementation of the present disclosure.

FIG. 5 depicts Raman spectra of ODF containing Vitamin B12 prepared using double screw hot melt extrusion in accordance with an implementation of the present disclosure.

FIG. 6 illustrates the dissolution studies of the oral dispersible composition in accordance with an implementation of the present disclosure.

FIG. 7 displays the measurement of the contact angle for evaluation of oral dispersible film composition in accordance with an implementation of the present disclosure.

FIG. 8 shows a gas chromatogram of the composition in accordance with an implementation of the present disclosure.

FIG. 9 shows the X-ray diffraction studies in accordance with an implementation of the present disclosure.

FIG. 10 displays the thermo-gravimetric analysis of the composition in accordance with an implementation of the present disclosure.

FIG. 11a displays the High Performance Liquid Chromatography (HPLC) analysis of standard compound. FIG. 11b displays the high performance liquid chromatography (HPLC) analysis of assay compound in the composition in accordance with an implementation of the present disclosure.

FIG. 12 displays the culture plate depicting the microbiological viability assay for a standard in accordance with an implementation of the present disclosure.

FIG. 13 displays the culture plate depicting the microbiological viability assay for a sample in accordance with an implementation of the present disclosure.

FIG. 14 displays the culture plate depicting the microbiological viability assay for Lactobacillus acidophilus in accordance with an implementation of the present disclosure.

FIG. 15 displays the excipient computability for Lactobacillus acidophilus with films in accordance with an implementation of the present disclosure.

FIG. 16a displays the High Performance Liquid Chromatography (HPLC) analysis of standard compound. FIG. 16b displays the High Performance Liquid Chromatography (HPLC) analysis of assay compound in the composition in accordance with an implementation of the present disclosure.

FIG. 17 illustrates the dissolution studies of the oral dispersible composition in accordance with an implementation of the present disclosure.

FIG. 18 illustrates the ex vivo mucoadhesive test of the oral dispersible composition in accordance with an implementation of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” is used to mean one or more and thus includes individual components as well as mixtures/combinations.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature ranges of about 25-40° C. should be interpreted to include not only the explicitly recited limits of about 25° C. to about 40° C., but also to include sub-ranges, such as 25-30° C., 28-38° C., and so forth, as well as individual amounts, including fractional amounts, within the specified ranges, such as 25.2° C., and 38.5° C., for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

“Hot melt extrusion” (HME) is the process of applying heat and pressure to melt a polymer and force it though an orifice in a continuous process to obtain the product/delivery systems. HME is carried out using an extruder—a barrel containing one (single screw) or two co/counter-rotating screws (twin-screw) that transport material down the barrel.

The term “oral dispersible film” composition denotes to a composition that is in the form of a thin film made up of from a definite ratio of film forming polymers and a plasticizer to provide a smooth texture and uniform thickness. The oral dispersible film composition essentially comprises an active ingredient which is dispersed uniformly through the film. The inform distribution of the active and the desired texture of the film is achieved by using a twin-screw hot melt extrusion process.

The present disclosure is not to be limited in scope by the specific implementations described herein, which are intended for the purposes of exemplification only. Functionally-equivalent products, compositions, and methods are clearly within the scope of the disclosure, as described herein.

As discussed above, the oral dispersible films are easier and convenient to use and those prepared via hot melt extrusion process are environmental friendly too. Therefore, an oral dispersible film composition has been described herein.

In an embodiment of the present disclosure, there is provided an oral dispersible film composition comprising: (a) maltodextrin; and (b) hydroxypropyl cellulose, wherein the weight ratio of maltodextrin to hydroxypropyl cellulose is in the range of 1:1-3:1. In another embodiment of the present disclosure the weight ratio of maltodextrin to hydroxypropyl cellulose is in the range of 1.2:1-2.8:1. In another embodiment of the present disclosure the weight ratio of maltodextrin to hydroxypropyl cellulose is in the range of 1.5:1-2.5:1.

In an embodiment of the present disclosure, there is provided an oral dispersible film composition as described herein, wherein the at least one plasticizer is selected from the group consisting of sorbitol, glycerol, polyethylene glycol (PEG), triethylcitrate (TEC), propylene glycol (PG), and combinations thereof.

In an embodiment of the present disclosure, there is provided an oral dispersible film composition as described herein, wherein the at least one plasticizer is sorbitol.

In an embodiment of the present disclosure, there is provided an oral dispersible film composition comprising: (a) maltodextrin; (b) hydroxypropyl cellulose; and (c) plasticizer, wherein the weight ratio of maltodextrin to hydroxypropyl cellulose is in the range of 1:1-3:1.

In an embodiment of the present disclosure, there is provided an oral dispersible film composition as described herein, wherein the weight ratio of maltodextrin to hydroxypropyl cellulose to plasticizer is in the range of 3:1:1-3:1:3. In another embodiment of the present disclosure the weight ratio of maltodextrin to hydroxypropyl cellulose to plasticizer is in the range of 3:1:1.2-3:1:2.8. In another embodiment of the present disclosure the weight ratio of maltodextrin to hydroxypropyl cellulose to plasticizer is in the range of 3:1:1.5-3:1:2.5.

In an embodiment of the present disclosure, there is provided an oral dispersible film composition comprising: (a) maltodextrin; (b) hydroxypropyl cellulose; and (c) plasticizer, wherein the weight ratio of maltodextrin to hydroxypropyl cellulose is in the range of 1:1-3:1, and the weight ratio of maltodextrin to hydroxypropyl cellulose to plasticizer is in the range of 3:1:1-3:1:3.

In an embodiment of the present disclosure, there is provided an oral dispersible film composition as described herein, wherein the oral dispersible film composition comprises at least one active ingredient dispersed in a matrix comprising the oral dispersible film composition.

In an embodiment of the present disclosure, there is provided an oral dispersible film composition comprising: (a) maltodextrin; (b) hydroxypropyl cellulose; (c) plasticizer; and (d) at least one active ingredient dispersed in a matrix comprising the oral dispersible film composition, wherein the weight ratio of maltodextrin to hydroxypropyl cellulose to plasticizer is in the range of 3:1:1-3:1:3.

In an embodiment of the present disclosure, there is provided an oral dispersible film composition comprising: (a) maltodextrin; (b) hydroxypropyl cellulose; (c) plasticizer; and (d) at least one active ingredient, wherein the at least one active ingredient is dispersed in a matrix comprising maltodextrin, hydroxypropyl cellulose and at least one plasticizer, and the weight ratio of maltodextrin to hydroxypropyl cellulose to plasticizer is in the range of 3:1:1-3:1:3.

In an embodiment of the present disclosure, there is provided an oral dispersible film composition as described herein, wherein the at least one active ingredient selected from the group consisting of mouth freshener, pharmaceutical agent, probiotics, dietary supplement, antioxidant, and combinations thereof.

In an embodiment of the present disclosure, there is provided an oral dispersible film composition as described herein, wherein at least one active ingredient is selected from the group consisting of menthol, eucalyptol, methyl salicylate, thymol, cocoa powder, folic acid, vitamin B6 (active form—pyridoxal 5′-phosphate), lipoic acid, vitamin B12 (cobalamin), (3-carotene, calcium, vitamin D3 (cholecalciferol), vitamin C (ascorbic acid), green tea, caffeine, ondansetron hydrochloride, memantine hydrochloride, isosorbide dinitrate, risperidone, diphenhydramine hydrochloride, ibuprofen, benzocaine, probiotics, brompheniramine maleate, chlorpheniramine maleate, carbinoxamine maleate, clemastine fumarate, dexchlorpheniramine maleate, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, loratadine, brompheniramine, dexbropheniramine, fexofenadine, cetirizine, famotidine, ranitidine, aspirin, acetaminophen, ketoprofen, diflunisal, fenoprofen, naproxen, tolmetin sodium, indomethacin, flurbiprofen sodium, celecoxib, valdecoxib, rofecoxib, sildenafil citrate, tadalafil, catechu, and combinations thereof.

In an embodiment of the present disclosure, there is provided an oral dispersible film composition comprising: (a) maltodextrin; (b) hydroxypropyl cellulose; (c) plasticizer; and (d) at least one active ingredient selected from the group consisting of mouth freshener, pharmaceutical agent, probiotics, dietary supplement, antioxidant, and combinations thereof, wherein the weight ratio of maltodextrin to hydroxypropyl cellulose to plasticizer is in the range of 3:1:1-3:1:3.

In an embodiment of the present disclosure, there is provided an oral dispersible film composition as described herein, wherein the oral dispersible film composition has an average thickness in the range of 0.04-0.8 mm.

In an embodiment of the present disclosure, there is provided an oral dispersible film composition as described herein, wherein the oral dispersible film composition has an average thickness in the range of 0.06-0.7 mm.

In an embodiment of the present disclosure, there is provided an oral dispersible film composition comprising: (a) maltodextrin; (b) hydroxypropyl cellulose; (c) plasticizer; and (d) at least one active ingredient, wherein the weight ratio of maltodextrin to hydroxypropyl cellulose is in the range of 1:1-3:1, and the oral dispersible film composition has an average thickness in the range of 0.04-0.8 mm.

In an embodiment of the present disclosure, there is provided an oral dispersible film composition comprising: (a) maltodextrin; (b) hydroxypropyl cellulose; (c) plasticizer; and (d) at least one active ingredient, wherein the weight ratio of maltodextrin to hydroxypropyl cellulose to plasticizer is in the range of 3:1:1-3:1:3, the oral dispersible film composition has an average thickness in the range of 0.04-0.8 mm, and the at least one active ingredient is dispersed in a matrix comprising the oral dispersible film composition.

In an embodiment of the present disclosure, there is provided an oral dispersible film composition as described herein, wherein the oral dispersible film composition is a fast dissolving film exhibiting an in-vitro release of at least 60% w/w in 1-2 minutes.

In an embodiment of the present disclosure, there is provided an oral dispersible film composition comprising: (a) maltodextrin; (b) hydroxypropyl cellulose; (c) plasticizer; and (d) at least one active ingredient, wherein the weight ratio of maltodextrin to hydroxypropyl cellulose is in the range of 1:1-3:1, and the oral dispersible film composition is a fast dissolving film exhibiting an in-vitro release of at least 60% w/w in 1-2 minutes.

In an embodiment of the present disclosure, there is provided an oral dispersible film composition comprising: (a) maltodextrin; (b) hydroxypropyl cellulose; (c) plasticizer; and (d) at least one active ingredient, wherein the weight ratio of maltodextrin to hydroxypropyl cellulose to plasticizer is in the range of 3:1:1-3:1:3, the oral dispersible film composition is a fast dissolving film exhibiting an in-vitro release of at least 60% w/w in 1-2 minutes, and the at least one active ingredient is dispersed in a matrix comprising the oral dispersible film composition.

In an embodiment of the present disclosure, there is provided an oral dispersible film composition comprising: (a) at least one active ingredient has a weight percentage in the range of 0.01-45% with respect to the composition and is selected from the group consisting of menthol, vitamin B6, folic acid, lipoic acid, β-carotene, vitamin B12, cocoa powder, calcium, vitamin D3, vitamin C, green tea, caffeine, ondansetron hydrochloride, memantine hydrochloride, isosorbide dinitrate, risperidone, diphenhydramine hydrochloride, ibuprofen, benzocaine, probiotics, brompheniramine maleate, chlorpheniramine maleate, carbinoxamine maleate, clemastine fumarate, dexchlorpheniramine maleate, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, loratadine, brompheniramine, dexbropheniramine, fexofenadine, cetirizine, famotidine, ranitidine, aspirin, acetaminophen, ketoprofen, diflunisal, fenoprofen, naproxen, tolmetin sodium, indomethacin, flurbiprofen sodium, celecoxib, valdecoxib, rofecoxib, sildenafil citrate, tadalafil, catechu, and combinations thereof; (b) maltodextrin has a weight percentage in the range of 28-65% with respect to the composition; (c) hydroxypropyl cellulose has a weight percentage in the range of 10-30% with respect to the composition; and (d) at least one plasticizer has a weight percentage in the range of 1-15% with respect to the composition, wherein the weight ratio of maltodextrin to hydroxypropyl cellulose to plasticizer is in the range of 3:1:1-3:1:3.

In an embodiment of the present disclosure, there is provided an oral dispersible film composition comprising: (a) at least one active ingredient has a weight percentage in the range of 0.25-40% with respect to the composition and is selected from the group consisting of menthol, vitamin B6, folic acid, lipoic acid, β-carotene, vitamin B12, cocoa powder, calcium, vitamin D3, vitamin C, green tea, caffeine, ondansetron hydrochloride, memantine hydrochloride, isosorbide dinitrate, risperidone, diphenhydramine hydrochloride, ibuprofen, benzocaine, probiotics, brompheniramine maleate, chlorpheniramine maleate, carbinoxamine maleate, clemastine fumarate, dexchlorpheniramine maleate, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, loratadine, brompheniramine, dexbropheniramine, fexofenadine, cetirizine, famotidine, ranitidine, aspirin, acetaminophen, ketoprofen, diflunisal, fenoprofen, naproxen, tolmetin sodium, indomethacin, flurbiprofen sodium, celecoxib, valdecoxib, rofecoxib, sildenafil citrate, tadalafil, catechu, and combinations thereof; (b) maltodextrin has a weight percentage in the range of 29-58% with respect to the composition; (c) hydroxypropyl cellulose has a weight percentage in the range of 12-26% with respect to the composition; and (d) at least one plasticizer has a weight percentage in the range of 2-14% with respect to the composition, wherein the weight ratio of maltodextrin to hydroxypropyl cellulose to plasticizer is in the range of 3:1:1-3:1:3.

In an embodiment of the present disclosure, there is provided an oral dispersible film composition as described herein, optionally comprising at least one saliva stimulating agent selected from the group consisting of citric acid, sodium croscarmellose, tartaric acid, lactic acid, ascorbic acid, malic acid, and combinations thereof, and having a weight percentage in the range of 0.1-5% with respect to the composition. In another embodiment of the present disclosure at least one saliva stimulating agent has a weight percentage in the range of 0.5-4.5% with respect to the composition. In another embodiment of the present disclosure at least one saliva stimulating agent has a weight percentage in the range of 1.0-4.0% with respect to the composition.

In an embodiment of the present disclosure, there is provided an oral dispersible film composition comprising: (a) maltodextrin; (b) hydroxypropyl cellulose; (c) plasticizer; (d) at least one active ingredient; and (e) at least one saliva stimulating agent, wherein the weight ratio of maltodextrin to hydroxypropyl cellulose is in the range of 1:1-3:1, and the weight ratio of maltodextrin to hydroxypropyl cellulose to plasticizer is in the range of 3:1:1-3:1:3.

In an embodiment of the present disclosure, there is provided an oral dispersible film composition comprising: (a) maltodextrin; (b) hydroxypropyl cellulose; (c) plasticizer; (d) at least one active ingredient; and (e) at least one saliva stimulating agent, wherein the weight ratio of maltodextrin to hydroxypropyl cellulose is in the range of 1:1-3:1, and the oral dispersible film composition has an average thickness in the range of 0.04-0.8 mm.

In an embodiment of the present disclosure, there is provided an oral dispersible film composition comprising: (a) maltodextrin; (b) hydroxypropyl cellulose; (c) plasticizer; (d) at least one active ingredient; and (e) at least one saliva stimulating agent, wherein the weight ratio of maltodextrin to hydroxypropyl cellulose to plasticizer is in the range of 3:1:1-3:1:3, the oral dispersible film composition has an average thickness in the range of 0.04-0.8 mm, and the at least one active ingredient is dispersed in a matrix comprising the oral dispersible film composition.

In an embodiment of the present disclosure, there is provided an oral dispersible film composition comprising: (a) maltodextrin; (b) hydroxypropyl cellulose; (c) plasticizer; (d) at least one active ingredient; and (e) at least one saliva stimulating agent, wherein the weight ratio of maltodextrin to hydroxypropyl cellulose is in the range of 1:1-3:1, and the oral dispersible film composition is a fast dissolving film exhibiting an in-vitro release of at least 60% w/w in 1-2 minutes.

In an embodiment of the present disclosure, there is provided an oral dispersible film composition comprising: (a) maltodextrin; (b) hydroxypropyl cellulose; (c) plasticizer; (d) at least one active ingredient; and (e) at least one saliva stimulating agent, wherein the weight ratio of maltodextrin to hydroxypropyl cellulose to plasticizer is in the range of 3:1:1-3:1:3, the oral dispersible film composition is a fast dissolving film exhibiting an in-vitro release of at least 60% w/w in 1-2 minutes, and the at least one active ingredient is dispersed in a matrix comprising the oral dispersible film composition.

In an embodiment of the present disclosure, there is provided an oral dispersible film composition comprising: (a) maltodextrin; (b) hydroxypropyl cellulose; (c) plasticizer; (d) at least one active ingredient; and (e) at least one saliva stimulating agent, wherein the weight ratio of maltodextrin to hydroxypropyl cellulose to plasticizer is in the range of 3:1:1-3:1:3, the oral dispersible film composition has an average thickness in the range of 0.04-0.8 mm, the oral dispersible film composition is a fast dissolving film exhibiting an in-vitro release of at least 60% w/w in 1-2 minutes, and the at least one active ingredient is dispersed in a matrix comprising the oral dispersible film composition.

In an embodiment of the present disclosure, there is provided an oral dispersible film composition as described herein, optionally comprising at least one pharmaceutically acceptable excipient selected from the group consisting of anti-sticking agent, sweetener, preservative, lubricant, flavoring agent, mucoadhesive agent, and combinations thereof.

In an embodiment of the present disclosure, there is provided an oral dispersible film composition comprising: (a) maltodextrin; (b) hydroxypropyl cellulose; (c) plasticizer; (d) at least one active ingredient; (e) at least one saliva stimulating agent; and (f) excipient, wherein the weight ratio of maltodextrin to hydroxypropyl cellulose is in the range of 1:1-3:1, and the weight ratio of maltodextrin to hydroxypropyl cellulose to plasticizer is in the range of 3:1:1-3:1:3.

In an embodiment of the present disclosure, there is provided an oral dispersible film composition as described herein, wherein the anti-sticking agent has a weight percentage in the range of 0.1-8.0% with respect to the composition.

In an embodiment of the present disclosure, there is provided an oral dispersible film composition as described herein, wherein the anti-sticking agent has a weight percentage in the range of 0.1-7.4% with respect to the composition. In another embodiment of the present disclosure the anti-sticking agent has a weight percentage in the range of 0.1-0.5% with respect to the composition. In another embodiment of the present disclosure the anti-sticking agent has a weight percentage in the range of 0.2-0.4% with respect to the composition.

In an embodiment of the present disclosure, there is provided an oral dispersible film composition as described herein, wherein the anti-sticking agent is selected from the group consisting of microcrystalline cellulose, sodium lauryl sulfate, silicon dioxide, tween, polyoxyethylene alkylethers, bezthonium chloride, dibutyl tartrate, and combinations thereof.

In an embodiment of the present disclosure, there is provided an oral dispersible film composition as described herein, wherein the anti-sticking agent is microcrystalline cellulose.

In an embodiment of the present disclosure, there is provided an oral dispersible film composition as described herein, wherein the sweetener has a weight percentage in the range of 0.1-5.0% with respect to the composition.

In an embodiment of the present disclosure, there is provided an oral dispersible film composition as described herein, wherein the sweetener has a weight percentage in the range of 0.1-2.0% with respect to the composition.

In an embodiment of the present disclosure, there is provided an oral dispersible film composition as described herein, wherein the sweetener is selected from the group consisting of sucralose, xylose, ribose, glucose, mannose, galactose, fructose, dextrose, sucrose, maltose, partially hydrolyzed starch, corn syrup solids, xylitol, mannitol, saccharin salts, cyclamate salts, acesulfam-K, aspartame, neotame, and combinations thereof.

In an embodiment of the present disclosure, there is provided an oral dispersible film composition as described herein, wherein the sweetener is sucralose.

In an embodiment of the present disclosure, there is provided an oral dispersible film composition as described herein, wherein the preservative has a weight percentage in the range of 0.05-3.0% with respect to the composition.

In an embodiment of the present disclosure, there is provided an oral dispersible film composition as described herein, wherein the preservative has a weight percentage in the range of 0.05-2.0% with respect to the composition.

In an embodiment of the present disclosure, there is provided an oral dispersible film composition as described herein, wherein the preservative is selected from the group consisting of sodium benzoate, benzalkonium chloride, potassium sorbate, methyl paraben, propyl paraben, and combinations thereof.

In an embodiment of the present disclosure, there is provided an oral dispersible film composition as described herein, wherein the preservative is sodium benzoate, benzalkonium chloride, and combinations thereof.

In an embodiment of the present disclosure, there is provided an oral dispersible film composition as described herein, wherein the lubricant has a weight percentage in the range of 0.1-1% with respect to the composition.

In an embodiment of the present disclosure, there is provided an oral dispersible film composition as described herein, wherein the lubricant has a weight percentage in the range of 0.1-0.5% with respect to the composition.

In an embodiment of the present disclosure, there is provided an oral dispersible film composition as described herein, wherein the lubricant is selected from the group consisting of sodium hyaluronate, sucrose esters, glyceryl behenate (stelliesters), stearic acid, dimethyl phthalate, diethyl phthalate, dibutyl phthalate, tributyl citrate, triethyl citrate, acetyl citrate, triacetin, dioctyl adipate, diethyl adipate, di(2-methylethyl) adipate, dihexyl adipate, partial fatty acid esters of sugars, polyethylene glycol fatty acid esters, polyethylene glycol fatty alcohol ethers, polyethylene glycol sorbitan fatty acid esters, 2-ethoxy ethanol, ethyl alcohol, propyl alcohol, butyl alcohol, pentyl alcohol, hexyl alcohol, heptyl alcohol, octyl alcohol, dibutyl tartrate, castor oil, and combinations thereof.

In an embodiment of the present disclosure, there is provided an oral dispersible film composition as described herein, wherein the lubricant is sodium hyaluronate.

In an embodiment of the present disclosure, there is provided an oral dispersible film composition as described herein, wherein the flavoring agent has a weight percentage in the range of 0.5-6% with respect to the composition.

In an embodiment of the present disclosure, there is provided an oral dispersible film composition as described herein, wherein the flavoring agent has a weight percentage in the range of 0.7-5% with respect to the composition.

In an embodiment of the present disclosure, there is provided an oral dispersible film composition as described herein, wherein the flavoring agent is selected from the group consisting of orange flavor, green mango flavor, cocoa flavor, banana flavor, strawberry flavor, custard apple flavor, watermelon flavor, pineapple flavor and various fruit flavors, mint flavor, peppermint flavor, and combinations thereof.

In an embodiment of the present disclosure, there is provided an oral dispersible film composition as described herein, wherein the flavoring agent is selected from the group consisting of orange flavor, green mango flavor, cocoa flavor, and combinations thereof.

In an embodiment of the present disclosure, there is provided an oral dispersible film composition as described herein, wherein the mucoadhesive agent has a weight percentage in the range of 0.1-8.0%.

In an embodiment of the present disclosure, there is provided an oral dispersible film composition as described herein, wherein the mucoadhesive agent is selected from the group consisting of sodium alginate, sodium carboxymethyl cellulose, and combinations thereof.

In an embodiment of the present disclosure, there is provided a process for the preparation of oral dispersible film composition comprising (a) maltodextrin; and (b) hydroxypropyl cellulose, wherein the weight ratio of maltodextrin to hydroxypropyl cellulose is in the range of 1:1-3:1, said process comprising the steps of: (a) contacting maltodextrin, hydroxypropyl cellulose, plasticizer, at least one active ingredient, at least one saliva stimulating agent, and excipient to obtain a first mixture; and (b) extruding the first mixture to obtain the oral dispersible film composition.

In an embodiment of the present disclosure, there is provided a process for the preparation of oral dispersible film composition comprising the steps of: (a) contacting at least one active ingredient, maltodextrin, hydroxypropyl cellulose, at least one saliva stimulating agent, sweetener, and flavoring agent to obtain a first mixture; (b) contacting the first mixture, plasticizer, and anti-sticking agent to obtain a second mixture; and (c) extruding the second mixture to obtain the oral dispersible film composition.

In an embodiment of the present disclosure, there is provided a process for the preparation of oral dispersible film composition comprising (a) maltodextrin; and (b) hydroxypropyl cellulose, wherein the weight ratio of maltodextrin to hydroxypropyl cellulose is in the range of 1:1-3:1, said process comprising the steps of: (a) contacting at least one active ingredient, maltodextrin, hydroxypropyl cellulose, at least one saliva stimulating agent, sweetener, and flavoring agent to obtain a first mixture; (b) contacting the first mixture, plasticizer, and anti-sticking agent to obtain a second mixture; and (c) extruding the second mixture to obtain the oral dispersible film composition.

In an embodiment of the present disclosure, there is provided a process for the preparation of oral dispersible film composition comprising (a) maltodextrin; (b) hydroxypropyl cellulose and (c) plasticizer, wherein the weight ratio of maltodextrin to hydroxypropyl cellulose is in the range of 1:1-3:1, said process comprising the steps of: (a) contacting at least one active ingredient, maltodextrin, hydroxypropyl cellulose, at least one saliva stimulating agent, sweetener, and flavoring agent to obtain a first mixture; (b) contacting the first mixture, plasticizer, and anti-sticking agent to obtain a second mixture; and (c) extruding the second mixture to obtain the oral dispersible film composition, wherein the weight ratio of maltodextrin to hydroxypropyl cellulose to plasticizer is in the range of 3:1:1-3:1:3.

In an embodiment of the present disclosure, there is provided a process for the preparation of oral dispersible film composition comprising (a) maltodextrin; (b) hydroxypropyl cellulose, (c) plasticizer, (d) at least one active, (e) at least one saliva stimulating agent, (f) sweetener, (g) flavouring agent and (h) anti-sticking agent, wherein the weight ratio of maltodextrin to hydroxypropyl cellulose is in the range of 1:1-3:1, said process comprising the steps of: (a) contacting at least one active ingredient, maltodextrin, hydroxypropyl cellulose, at least one saliva stimulating agent, sweetener, and flavoring agent to obtain a first mixture; (b) contacting the first mixture, plasticizer, and anti-sticking agent to obtain a second mixture; and (c) extruding the second mixture to obtain the oral dispersible film composition, wherein the weight ratio of maltodextrin to hydroxypropyl cellulose to plasticizer is in the range of 3:1:1-3:1:3.

In an embodiment of the present disclosure, there is provided a process for the preparation of oral dispersible film composition as described herein, wherein extruding the second mixture is carried out through a twin screw hot melt extrusion method.

In an embodiment of the present disclosure, there is provided a process for the preparation of oral dispersible film composition as described herein, wherein extruding the second mixture is carried out through a twin screw hot melt extrusion method with a screw speed in the range of 30-90 rpm.

In an embodiment of the present disclosure, there is provided a process for the preparation of oral dispersible film composition as described herein, wherein extruding the second mixture is carried out at a temperature in the range of 80-110° C. to obtain the oral dispersible film composition. In another embodiment of the present disclosure extruding the second mixture is carried out at a temperature in the range of 85-105° C. to obtain the oral dispersible film composition. In another embodiment of the present disclosure extruding the second mixture is carried out at a temperature in the range of 90-100° C. to obtain the oral dispersible film composition.

In an embodiment of the present disclosure, there is provided a system for the preparation of oral dispersible film composition comprising (a) maltodextrin; (b) hydroxypropyl cellulose and (c) plasticizer, wherein the weight ratio of maltodextrin to hydroxypropyl cellulose is in the range of 1:1-3:1, said system comprising: (a) a hopper zone for feeding the second mixture as described herein; (b) a conveying zone for transporting, processing and homogenizing the second mixture; (c) an orifice zone for shaping the homogenized second mixture to obtain a film of the second mixture; and (d) a downstream ancillary equipment zone for collecting the film of the oral dispersible film composition, wherein the oral dispersible film composition is in the form of a film having an average thickness in the range of 0.04-0.8 mm.

In an embodiment of the present disclosure, there is provided a system for the preparation of oral dispersible film composition comprising: (a) a hopper zone for feeding the second mixture as described herein; (b) a conveying zone for transporting, processing and homogenizing the second mixture; (c) an orifice zone for shaping the homogenized second mixture to obtain a film of the second mixture; and (d) a downstream ancillary equipment zone for collecting the film of the oral dispersible film composition, wherein the oral dispersible film composition is in the form of a film having an average thickness in the range of 0.04-0.8 mm, and exhibiting an in-vitro release of at least 60% w/w in 1-2 minutes.

In an embodiment of the present disclosure, there is provided a process for the preparation of oral dispersible film composition as described herein, wherein extruding the second mixture comprises the steps of: (a) feeding the second mixture in the hopper zone; (b) processing the second mixture to obtain the oral dispersible film composition in the conveying zone; (c) shaping the oral dispersible film composition obtained from the conveying zone into a film in the orifice zone; and (d) cooling and collecting the film of the oral dispersible film composition from the downstream ancillary equipment zone.

Despite the fact that the subject matter of the present disclosure has been described in considerable detail with reference to some preferred embodiments, various other embodiments are also possible.

### EXAMPLES

The working examples of the present disclosure would now be illustrated. These examples are neither restrictive nor intended to be limiting to the scope of the present invention. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### Formulation Development

The development of the composition for the oral dispersible films was done by selecting and analysing various constituents of the composition on certain parameters.

### Selection of Polymer:

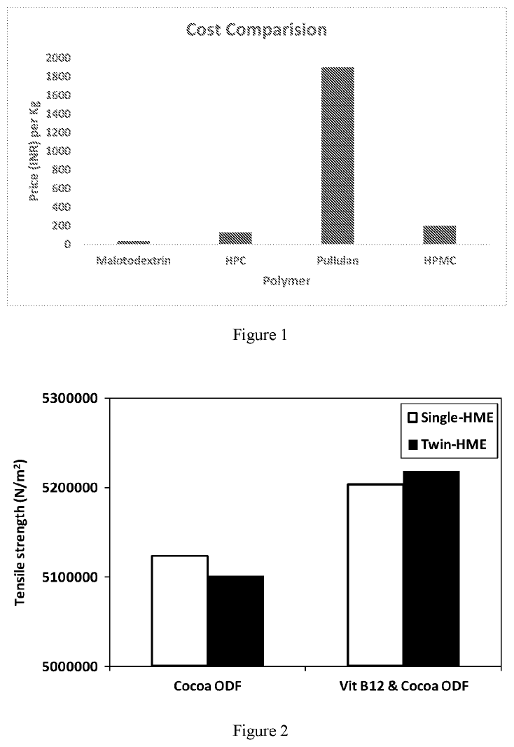

Polymers are the most important ingredient of the oral fast dissolving film. These polymers have mostly attracted considerable attention by medical and nutraceutical industry. The polymers can be used alone or in combination with other polymers to obtain the desired strip properties. The film obtained should be tough enough so that there won't be any damage while handling or during transportation. The robustness of the strip depends on the type of polymer and their amount in the formulation (FIG. 1). Several analyses were performed on various polymers and maltodextrin (MDX) was found to be the cheapest and most effective film forming molecule.

A combination of polymers was chosen for making the film of the ODFs. Hydroxypropyl cellulose (HPC), hydroxypropyl methyl cellulose (HPMC) and polyvinyl pyrrolidone (PVP) were screened in combination with maltodextrin for testing their film forming capability.

Table 1 displays the development of the polymer film evaluating various combinations of different polymers (in definite ratios) with maltodextrin.

TABLE 1
Details of optimization ratio (maltodextrin:different polymers).

| | |
|---|---|
| Ratio | Physical characteristics |

MDX:HPMC

| | |
|---|---|
| 9:1 | Thick films were formed with low folding |
| 8:1 | endurance |
| 7:1 | |
| 6:1 | |
| 5:1 | |
| 4:1 | |
| 3:1 | |
| 2:1 | |
| 1:1 | |

MDX:PVP

| | |
|---|---|
| 9:1 | Thick and brittle films |
| 8:1 | |
| 7:1 | |
| 6:1 | |
| 5:1 | |
| 4:1 | Very Low folding endurance |
| 3:1 | |
| 2:1 | Soft and flexible but low folding endurance |
| 1:1 | Soft films but not flexible |

MDX:HPC (low viscosity)

| | |
|---|---|
| 9:1 | Thick and brittle films |
| 8:1 | |
| 7:1 | Soft, flexible films and very low folding |
| 6:1 | endurance |
| 5:1 | |
| 4:1 | Soft, flexible films and Low folding |
| | endurance |
| 3:1 | Soft and flexible film with good folding |
| 2:1 | endurance and tensile strength |
| 1:1 | Soft and flexible film with high very |
| | folding endurance and tensile strength |

MDX = Maltodextrin
HPMC = Hydroxypropyl methyl cellulose
PVP = Polyvinyl pyrrolidone
HPC = Hydroxypropyl cellulose

Disintegration of the film was found to be inversely dependent on concentration of the HPC polymer used in the formulation. Table 1 infers that a combination of maltodextrin and hydroxypropyl cellulose with a ratio in the range of 1:1-3:1 results in the formation of desired film with high tensile strength and folding endurance.

The regulation of temperature is a crucial aspect of film extrusion using a hot melt extruder. Therefore, the range of temperature that can be used to form an appropriate and desired film from the hot melt extruder was optimized using maltodextrin. The optimum range of temperature for extrusion of polymer film was optimized to be in the range of 75-110° C., as displayed in Table 2 below.

TABLE 2
Temperature optimization for film extrusion.

| Formulation | Temperature (° C.) | Observation |
|---|---|---|
| Maltodextrin | 65 | No extrude |
| | 75 | film was extruded |
| | 85 | |
| | 95 | The film was extruded but |
| | 100 | more crispy nature |
| | 105 | |
| | 110 | |
| | 115 | film was charred |
| | 120 | |

It was found that a temperature in the range of 75-85° C. would be appropriate for accommodating the effective extrusion of the oral dispersible film (inferred from Table 2). Further, a higher temperature can be optimized using combination of polymer or adding plasticizing agent.

### Selection of Plasticizer:

The flow and texture of polymer was improved with the use of plasticizer that enhances the strength of the polymer. Glycerol, propylene glycol and other plasticizers were used for the purpose as displayed in Table 3 below.

The plasticizer employed should impart permanent flexibility (folding endurance) to the film which depends on the volatility of the plasticizer and the type of interaction with the polymer. Table 1 infers that a combination of maltodextrin and hydroxypropyl cellulose with a ratio in the range of 1:1-3:1 results in the formation of desired film with high tensile strength and folding endurance. However, a wider range of maltodextrin to HPC ratio from 1:7 to 1:4 was tested in combination with plasticizer to get desired film property (see table 3 below). maltodextrin and HPC were used in combination as the film forming polymers and the amount of plasticizer was optimized. Four plasticizers propylene glycol (PG), glycerol, triethylcitrate (TEC) and sorbitol were screened.

TABLE 3
Screening of plasticizers

| | |
|---|---|
| Ratio | Physical characteristics |

MDX:HPC:Propylene glycol (PG)

| | |
|---|---|
| 7:1:1 to 7:1:4 | Brittle film was formed, no tensile strength |
| 7:1:5 | Flexible sticky film was formed |
| 7:1:6 | |
| 6:1:1 | Brittle film was formed, no tensile strength |
| 6:1:2 | |
| 6:1:3 | Flexible film was formed but % elongation of |
| 5:1:1 | the film was less |
| 5:1:2 | |
| 5:1:3 | |
| 4:1:1 | % elongation was improved than previous trial |
| 4:1:2 | |
| 4:1:3 | Got desired flexibility and smooth texture of the |
| 3:1:1 | film, absorb moisture from the environment |
| 3:1:2 | |
| 3:1:3 | |

MDX:HPC:Glycerol

| | |
|---|---|
| 7:1:1 to 7:1:4 | Brittle film was formed, no tensile strength |
| 7:1:5 | Flexible sticky film was formed, no tensile |
| 7:1:6 | strength |
| 6:1:1 | |
| 6:1:2 | |
| 6:1:3 | |
| 5:1:1 | |
| 5:1:2 | |
| 5:1:3 | |
| 4:1:1 | Sticky ness of the film was improved than |
| 4:1:2 | previous trial |
| 4:1:3 | |
| 3:1:1 | Got desired flexibility and smooth texture of the |
| 3:1:2 | film, absorb moisture from environment |
| 3:1:3 | |

MDX:HPC:Triethyl citrate (TEC)

| | |
|---|---|
| 7:1:1 to 7:1:4 | No tensile strength, very less % elongation, |
| 7:1:5 | flexibility of the film was less. |
| 7:1:6 | |
| 6:1:1 | |
| 6:1:2 | |
| 6:1:3 | |
| 5:1:1 | |
| 5:1:2 | |
| 5:1:3 | |
| 4:1:1 | |
| 4:1:2 | |
| 4:1:3 | |
| 3:1:1 | |
| 3:1:2 | |
| 3:1:3 | |

MDX:HPC:Polyethylene glycol (PEG)

| | |
|---|---|
| 7:1:1 to 7:1:4 | No tensile strength, very less % elongation, |
| 7:1:5 | flexibility of the film was less. |
| 7:1:6 | |
| 6:1:1 | |
| 6:1:2 | |
| 6:1:3 | |
| 5:1:1 | |
| 5:1:2 | |
| 5:1:3 | |
| 4:1:1 | |
| 4:1:2 | |
| 4:1:3 | |
| 3:1:1 | |
| 3:1:2 | |
| 3:1:3 | |

MDX:HPC:Sorbitol

| | |
|---|---|
| 7:1:1 to 7:1:4 | Brittle film was formed, no tensile strength |
| 7:1:5 | Flexible sticky film was formed, no tensile |
| 7:1:6 | strength |
| 6:1:1 | |
| 6:1:2 | |
| 6:1:3 | |
| 5:1:1 | |
| 5:1:2 | |
| 5:1:3 | |
| 4:1:1 | Sticky ness of the film was improved than |
| 4:1:2 | previous trial |
| 4:1:3 | |
| 3:1:1 | Got desired flexibility and smooth texture of the |
| 3:1:2 | film, absorb less environmental moisture than |
| 3:1:3 | glycerol |

MDX = Maltodextrin
HPC = Hydoxypropyl cellulose

Table 3 illustrates the optimization of the ratio range of maltodextrin to hydroxypropyl cellulose to plasticizer. The optimization data shows that sorbitol and glycerol were found to be acceptable as plasticizers because they provided films with desired characteristics. Although, sorbitol as the plasticizer used in a ratio in the range of 3:1:1-3:1:3 was selected to be the best working example as it absorbed less moisture than glycerol. Sorbitol also has a dual functionality and can act as a sweetening agent. However, the concentration of plasticizer may vary according to the solid state (melting point, moisture absorbing property, etc.) nature of active and other excipients used in the composition.

### Selection of Saliva Stimulant:

The purpose of using saliva stimulating agents is to increase the rate of production of saliva that would aid in the faster disintegration of the rapid dissolving strip formulations. Citric acid and croscarmellose sodium were the most commonly used saliva stimulants and hence were screened on the basis of in-vitro disintegration time. The disintegrating time of the present oral dispersible film composition was found to be within 15 minutes as per the standards due to the optimization of the weight percentage of the saliva stimulating agent.

TABLE 4

| Ingredient | % Composition | Disintegration time |
|---|---|---|
| Citric acid | 10 | Less than 10 seconds |
| | 5 | |
| | 1 | 14 sec |
| Croscarmellose Na | 10 | 13 sec |
| | 5 | Greater than 15 sec |
| | 1 | |

The optimization studies carried out for different weight percentages of the saliva stimulating agent resulted in identifying citric acid as the appropriate saliva stimulating agent. It is clear from Table 4 above that citric acid showed better saliva stimulating properties at an optimum concentration of 1% w/w. Although higher concentration of citric acid also induced better disintegration, but were observed to impart an undesirable astringent taste to the film. On the other hand, croscarmellose sodium was found to show greater than 15 seconds disintegration time and therefore was unsuitable to be used as saliva stimulating agent.

### Selection of Flavoring Agent:

The acceptance of the oral disintegrating or dissolving formulation by an individual (consumer) depends on the initial flavor quality. The flavor felt in first few seconds in the mouth after the product has been consumed and the after taste of the formulation which lasts for at least about 5 minutes are the key factors for the acceptance of an oral composition. Different flavors can be added in ODF as green mango, cocoa, orange, lemon etc. For mouthfreshener ODF, sucralose an artificial sweetener was used whereas at least one active like menthol, thymol, methyl salicylate and eucalyptol was incorporated to impart the mint flavor and good mouth feel. The above mentioned components (actives/sweetener) play a dual role in the oral dispersible film composition.

The concentration of menthol, eucalyptol and sucralose was optimized according to the mouth feel, freshening effect and the formulation was checked for its effect on disintegration time (Table 5).

TABLE 5
Optimization of components of mouth freshener imparting flavor

| Ingredients | % composition | Disintegration time |
|---|---|---|
| Sucralose | 0.3 | Less than 10 sec |
| Menthol | 0.038 | |
| Eucalyptus oil | 0.092 | |
| Methyl salicylate | 0.056 | |
| Thymol | 0.092 | |

### Other Excipients:

Addition of the plasticizer to improve the texture, tensile strength and folding endurance of the film also imparts stickiness. In order to prevent the stickiness, some anti-sticking agents are used. Sodium Lauryl Sulfate (SLS) and Microcrystalline Cellulose (MCC) were optimized to prevent the sticking of films inside the packing. Preservatives like potassium sorbate, sodium benzoate, methyl and propyl paraben were also optimized along with the anti-sticking agents. The results of the optimization have been summarized in Table 6 below.

TABLE 6
Optimization of anti-sticking agents and preservatives.

| Ingredients | % composition |
|---|---|
| Potassium sorbate | 0.05-2 |
| Methyl paraben | 0.18 |
| Propyl paraben | 0.02 |
| Sodium benzoate | 0.01-3 |
| SLS | 0.05 |
| MCC | 0.5-8 |

From the above optimization, it was found that the working range of the anti-sticking agent would be 0.05-8% w/w and that of the preservative in the range of 0.01-3% w/w respectively.

Furthermore, the above-mentioned components were combined and working ranges were established for the various components. The summary of the working range and role of all the components of the composition have been summarized in Table 7 below.

TABLE 7

| Ingredient name | Role | Working range |
|---|---|---|
| Maltodextrin (MDX) | Polymer for film | 28-65% |
| | formation | |
| Hydroxypropyl methyl cellulose (HPC) | Polymer for film | 10-30% |
| | formation | |
| Sorbitol | Plasticizer | 1-15% |
| Citric acid | Saliva stimulating agent | 0.1-5% |
| Sucralose | Sweetener | 0.1-2% |
| Microcrystalline cellulose (MCC) | Anti-sticking agent | 0.1-0.5% |
| Sodium benzoate, Benzalkonium chloride | Preservative | 0.05-2% |
| Sodium alginate | Mucoadhesive agent | 0.1-8% |
| Sodium carboxymethyl cellulose | Mucoadhesive agent | 0.1-8% |
| Sodium hyaluronate | Anti canker active/ | 0.1-1% |
| | Lubricant | |
| Orange flavour, Green mango flavour, Cocoa | Flavoring agent | 0.5-6% |
| flavour | | |
| Benzocaine, Menthol, Eucalyptol, Methyl | Active ingredient | 0.01-45% |
| salicylate, Thymol, Cocoa powder, Folic acid, | | |
| Vitamin B6, Lipoic acid, Beta-carotene, | | |
| Vitamin B12, Calcium (Ca), Vitamin D3, | | |
| Vitamin C, Green Tea, Caffeine, Ondansetron | | |
| hydrochloride, Memantine hydrochloride, | | |
| Isosorbide dinitrate, Risperidone, | | |
| Diphenhydramine hydrochloride, Ibuprofen, | | |
| Probiotics | | |

### Example 2

TABLE 8
Examples (1-5) of the oral dispersible film compositions developed.

| | Example 1 | | | Example 4 | Example 5 |
|---|---|---|---|---|---|
| | Mouth | Example 2 | Example 3 | Food | Opthalmic |
| | Freshener | Chocolate | Anti-anaemic | Supplement | Supplement |
| Ingredient name | film | film | film | film | film |
| Maltodextrin (MDX) | 57.68 | 47.49 | 55.45 | 43.33 | 47.22 |
| Hydroxypropyl methyl | 25.64 | 21.1 | 24.64 | 19.26 | 20.99 |
| cellulose (HPC) | | | | | |
| Sorbitol | 8 | 14 | 14 | 14 | 14 |
| Citric acid | 0.8 | 0.8 | 0.8 | 0.8 | 0.8 |
| Sucralose | 0.5 | 0.5 | 0.5 | 0.5 | 0.7 |
| Microcrystalline | 0.3 | 0.3 | 0.3 | 0.3 | 0.3 |
| cellulose (MCC) | | | | | |
| Sodium benzoate | 0.8 | 0.8 | 0.8 | 0.8 | 0.8 |
| Menthol | 0.038 | | | | 0.6 |
| Eucalyptol | 0.034 | | | | |
| Methyl salicylate | 0.056 | | | | |
| Thymol | 0.093 | | | | |
| Cocoa powder | | 15 | | | |
| Folic acid | | | 0.8 | | |
| Vitamin B6 | | | 1.9 | | |
| Lipoic acid | | | | 20 | |
| Beta-carotene | | | | | 4.8 |
| Orange flavour | | | 0.8 | 1 | |

TABLE 9
Examples (6-10) of the oral dispersible film compositions developed.

| | Example 6 | | | | |
|---|---|---|---|---|---|
| | Vitamin | Example 7 | Example 8 | | |
| | B12 Food | Food | Taste | Example 9 | Example 10 |
| | Supplement | Supplement | masked | Vitamin | Vitamin C |
| Ingredient name | film | film | Ca Fim | D3 film | film |
| Maltodextrin (MDX) | 57.46 | 43.75 | 29.21 | 51.36 | 40.98 |
| Hydroxypropyl methyl | 25.53 | 19.44 | 12.98 | 22.83 | 18.21 |
| cellulose (HPC) | | | | | |
| Sorbitol | 14 | 14 | 14 | 12 | 8 |
| Citric acid | 0.8 | 0.8 | 0.8 | 0.8 | 0.8 |
| Sucralose | 0.8 | 0.6 | 0.9 | 0.9 | 0.9 |
| Microcrystalline | 0.3 | 0.3 | 0.3 | 0.3 | 0.3 |
| cellulose (MCC) | | | | | |
| Sodium benzoate | 0.8 | 0.8 | 0.8 | 0.8 | 0.8 |
| Cocoa powder | | 20 | | | |
| Vitamin B12 | 0.3 | 0.3 | | | |
| Calcium (Ca) | | | 40 | | |
| Vitamin D3 | | | | 10 | |
| Vitamin C | | | | | 25 |
| Orange flavour | | | 1 | | |
| Green mango flavour | | | | 1 | |
| Cocoa flavour | | | | | 5 |

TABLE 10
Examples (11-15) of the oral dispersible film compositions developed.

| | | | | | Example 15 |
|---|---|---|---|---|---|
| | | | | Example 14 | Anti- |
| | Example 11 | Example 12 | Example 13 | film for | hypertensive/ |
| | Antioxidant | Caffeine | Anti-vomit | Alzheimer's | Anti-anginal |
| Ingredient name | film | film | film | disease | film |
| Maltodextrin (MDX) | 42.36 | 45.83 | 52.92 | 50.67 | 47.07 |
| Hydroxypropyl | 18.83 | 20.36 | 23.52 | 22.52 | 20.93 |
| methyl cellulose | | | | | |
| (HPC) | | | | | |
| Sorbitol | 14 | 14 | 13.2 | 14 | 4 |
| Citric acid | 0.8 | 0.8 | 0.8 | 0.8 | 1.2 |
| Sucralose | 0.9 | 0.9 | 1.5 | 0.9 | 0.5 |
| Microcrystalline | 0.3 | 0.3 | 0.3 | 0.3 | 0.3 |
| cellulose (MCC) | | | | | |
| Sodium benzoate | 0.8 | 0.8 | 0.8 | 0.8 | 1 |
| Sodium alginate | 2 | 2 | | | |
| Menthol | | | 0.8 | | |
| Eucalyptol | | | 0.15 | | |
| Green Tea | 20 | | | | |
| Caffeine | | 15 | | | |
| Ondansetron | | | 5 | | |
| hydrochloride | | | | | |
| Memantine | | | | 10 | |
| hydrochloride | | | | | |
| Isosorbide dinitrate | | | | | 25 |
| Green mango flavour | | | 1 | | |

TABLE 11
Examples (16-21) of the oral dispersible film compositions developed

| | | | Example 18 | Example 19 | Example 20 | |
|---|---|---|---|---|---|---|
| | | Example 17 | NSAID (Non-steroidal | Mucoadhesive | Mucoadhesive | |
| | Example 16 | Anti-hystemic/ | anti-inflammatory | film for canker | film for canker | Example 21 |
| | Anti-psychotic | Anti-allergenic | drug) | sores/mouth | sores/mouth | Probiotic |
| Ingredient name | film | film | film | ulcers | ulcers | film |
| Maltodextrin | 57.42 | 53.05 | 42.62 | 45.81 | 46.1 | 31.5 |
| (MDX) | | | | | | |
| Hydroxypropyl | 25.52 | 23.5 | 18.94 | 20.36 | 20.49 | 14 |
| methyl cellulose | | | | | | |
| (HPC) | | | | | | |
| Sorbitol | 14 | 12 | 2 | 10.5 | 10 | 12.5 |
| Citric acid | 0.8 | 0.8 | 0.8 | | | 0.8 |
| Sucralose | 0.9 | 0.9 | 1.2 | 0.2 | 0.2 | 0.9 |
| Microcrystalline | 0.3 | 0.3 | 0.3 | 0.3 | 0.3 | 0.3 |
| cellulose (MCC) | | | | | | |
| Sodium benzoate | 0.8 | 0.8 | 0.8 | | | |
| Sodium alginate | | | | 10 | 10 | |
| Sodium | | | | 5 | 5 | |
| carboxymethyl | | | | | | |
| cellulose | | | | | | |
| Benzocaine | | | | 7.5 | 7.5 | |
| Benzalkonium | | | | 0.13 | | |
| chloride | | | | | | |
| Menthol | | | | 0.2 | 0.2 | |
| Risperidone | 0.25 | | | | | |
| Diphenhydramine | | 8.57 | | | | |
| hydrochloride | | | | | | |
| Ibuprofen | | | 33.33 | | | |
| Sodium | | | | | 0.2 | |
| hyaluronate | | | | | | |
| Probiotics | | | | | | 40 |

### Example 3

### Method of Formation of the Oral Dispersible Film Composition

A large container was taken to mix all the powdered ingredients. Maltodextrin, hydroxypropyl cellulose, saliva stimulating agent, sweetener, flavoring agent along with the actives were mixed in the container to obtain a first mixture. To the first mixture was added antisticking agent and plasticizer and mixed to obtain a second mixture. The second mixture was then extruded using twin-screw hot melt extrusion over temperature in the range of 80-110° C. (most appropriately 85-95° C.). The screw design of the extruder was adjusted such that it used two mixing zone which mix and soften the material at a temperature which help to improve uniformity of the material, two kneading zones to break down the particles, making it less viscous and helping in decreasing the disintegration time of the film and conveying zone passes the material towards die zone. The screw rotations per minute (rpm) were in the range of 30-90 rpm which helped to increase the residence time of the material in the screw barrel. The increase in residence time increases shearing stress thereby decreasing the viscosity of the film, making it uniform and smooth. A uniform product can thus be obtained with desired attributes of film thickness and film width by optimization of process, formulation and equipment parameters.

### Example 4

### Hot Melt Extrusion Method

Hot melt extrusion (HME) is the process of applying heat and pressure to melt a polymer and force it though an orifice in a continuous process to obtain the product/delivery systems. HME is carried out using an extruder—a barrel containing one (single screw) or two co/counter-rotating screws (twin-screw) that transport material down the barrel. Typical hot melt extruder consists of four distinct parts:
1. An opening/hopper though which material enters the barrel to be extruded, or that may be continuously supplied to in a controlled manner by one or more external feeder(s),
2. A conveying section (process section), which comprises the barrel and the screw(s) that transport, and where applicable, mix/homogenize the material,
3. An orifice/die for shaping the material as it leaves the extruder,
4. Downstream ancillary equipments for cooling, cutting and/or collecting the finished product.

The process section can be divided into different zones, as per the consumer's perception. The temperature of different zones can be selectively controlled as per the processing requirements of the material in the equipment. After processing of the material, it can be cooled in the last zone and collected from the die end. Newer versions of twin-screw extruders are equipped with vents to remove the formed gases formed during product processing, additional ports/openings to introduce additional material during the processing of the material in the process section.

The present setup as described herein affords the continuous manufacturing of water soluble polymers based oral dispersible films using twin-screw hot melt extrusion process. It also covers the manufacturing of sublingual films using twin-screw hot melt extrusion process. Both the delivery systems can be easily used for incorporating medicament/nutraceutical active/inactive ingredient or a probiotics. The formulations relating to pharmaceutical/nutraceutical applications were developed and evaluated for various parameters affecting the organoleptic, sensorial and therapeutic properties of the films formed.

### Example 5

### Evaluation of Oral Dispersible Film Composition

Various water soluble polymers were utilized for developing ODFs using single screw extrusion process. The developed formulations were evaluated for various parameters. Based on the evaluations, selected polymers were then used for preparing ODFs using twin-screw hot met melt extrusion (TSE/HME). These evaluation parameters are explained as follows:

### Thickness:

The thickness of the formulated ODF was measured using Mitutoyo absolute digital vernier calliper. This is essential to ascertain uniformity in thickness of the film throughout the process. The thickness of the film sample was measured at five different locations (centre and four corners), and the mean thickness was determined. The values reported below are mean of 5 repeated values. The values are summarized in Table 12 below.

TABLE 12
Evaluation of thickness of ODFs extruded
using twin screw hot melt extruder.

| Formulation | Thickness (mm) |
|---|---|
| Mouth freshener film (Example 1) | 0.16 ± 0.14 |
| Green tea powder film (Example 11) | 0.25 ± 0.23 |
| Vitamin B12 film (Example 6) | 0.20 ± 0.11 |
| Cocoa film (Example 2) | 0.35 ± 0.18 |

It was observed that the film extruded from single-screw hot melt extruder is non-uniform in the appearance. The formulations extruded using TSE is uniform in nature throughout the sample. This is due to the uniform mixing of the polymeric blend using twin-screw in extruder and feeding of mixture through gravimetric feeder. The mixing using single-screw is not as efficient as compared to that of twin-screw HME.

### Tensile Strength:

The mechanical testing of the films is followed by American Society for Testing and Materials (ASTM) standards. Tensile strength analysis with ASTM D882 was followed.

Tensile testing determines the amount of stress each material can sustain prior to failure as well as the amount of elongation at the time of failure. Each specimen measured 80 mm (length)×5 mm (width)×2 mm (thickness). The tests were conducted using Brookfield Engineering CT3 texture analyzer with tensile jig and with a crosshead speed of 1 mm/s. The films were placed in the tensile jig holder, parallel to the direction of analysis. The analysis was repeated in 10 samples each and the average values are reported. Tensile strength (N/m²) is a property which is characteristic for each material/ingredient.

FIG. 2 depicts the tensile strength of ODFs prepared using single and twin-screw HME. For discussion, cocoa films (Example 2) and Vitamin B12 with cocoa powder (Example 7) are presented. From FIG. 2, it can be observed that tensile strengths for single-screw and twin-screw extruded ODF containing cocoa powder is similar. Similar observation is seen for ODF containing Vitamin B12 and cocoa powder. With average tensile strength of 5161810 N/m², the films developed using HME are durable enough to sustain the physical or environmental shocks.

### Scanning Electron Microscopy (SEM):

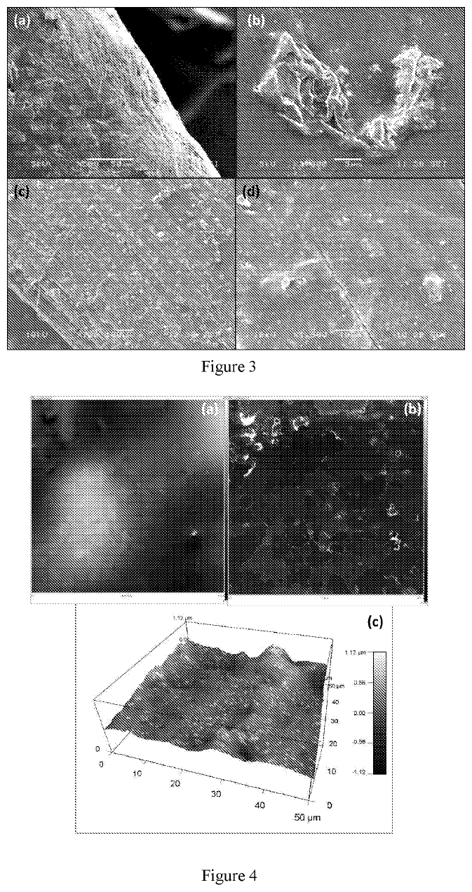

In order to observe the surface characteristics of ODFs made using single screw and twin-screw hot melt extrusion, film surface images were captured using SEM. FIGS. 3a and 3b depict the SEM images of ODF containing green tea, prepared using single screw hot melt extrusion at lower and higher magnification respectively. It can be observed from the FIG. 3a that the surface of the film is not uniform and uneven. FIG. 3b displays the small lumps present on the surface with many crevices. Hence, the films prepared using single screw extruder is not uniform throughout the sample, due to inefficient mixing of the polymeric blend in the equipment. FIGS. 3c and 3d portrays the SEM images of ODFs prepared using twin-screw hot melt extrusion. It can be seen from FIG. 3c that uniform vertical ridges are present throughout the film surface. These ridges have been formed during continuous manufacturing of the film to obtain uniform thickness by stretching the film on a conveyer belt. FIG. 3d is the 1500× magnified image of FIG. 3c and it depicts the uniformity of the films surface on higher magnification. Hence, the ODFs obtained using twin-screw hot melt extrusion were found to be comparatively smooth and uniform in film texture as compared to that of single screw extruded films.

### Atomic Force Microscopy (AFM):

AFM can be used to observe the topography of any various polymeric materials in their native environments. It is a non-destructive technique revealing details about the surface characteristics of the sample. It gives a 3D view of the surface topography which helps in better understanding of the surface structure. The ODFs developed using single screw and twin-screw HME were analyzed by AFM, with respect to their morphological surface interactions.

Samples were prepared by cutting freshly prepared ODFs with smooth surfaces (Cross section of extrudates) by a razor blade. All films with rectangular size of 20 mm×20 mm were selected, and placed on a petri dish. The flat bed of sample was then affixed on an optical glass slide by use of a 2-component epoxy resin, which get hardened within 3 min. The sample was placed such that it should keep its position horizontally, as this is required to obtain non-destructive imaging by atomic force microscope operations ⁽¹⁾. AFM analysis was carried out using AFM instrument of DFRT-PFM on a commercial SPM system (Asylum Research MFP-3D, California, USA) with a nitrogen flow cell positioned above an inverted optical microscope. film sample was mounted on glass on the micrometer positioning stage of a dimension icon of AFM with accelerating voltage up to ±220 V and imaging at ac voltages up to 110 Vpp (in the dual excitation mode) at frequencies of 300-400 kHz. Voltages were applied between the substrate and the conductive probe tips, and the current was recorded by the AFM's preamplifier (Asylum Research ORCA head model 59). The light source used in the AFM instrument is Super luminescent diode (SLD), classified as Class 1M light source. This enables polarization switching in film samples and imaging of the samples with maximum resolution and magnification. An AFM scans the surface of a specimen with a sharp tip mounted to a cantilever (Olympus TR400PB cantilevers), the deflections are directly related to the surface micro scale topography and its physical properties. About 20-30 regions per sample were scanned by the ‘programmed move’ in tapping mode. Height, phase and amplitude images were collected simultaneously using Platinum-coated, contact-mode AFM tips. Tips with diameter of less than 25 nm (Budget Sensors, BS-ElectriCont) were used. AFM images with areas of 10 mm×10 mm were recorded at higher resolution. All the AFM images were calculated from at least 20 images on each sample, illustrating the morphological surface interactions in detail. The resultant data was processed using Open user interface based on IGOR Pro software with OpenGL® 3D for advanced image display.

FIGS. 4a and 4b depicts the molecular fracture roughness images of the ODFs developed using single screw while 3c explains the 3D surface image, prepared using twin-screw HME. From FIGS. 4a and 4b, it can be observed that the surface of the mouth freshener film was rough and not uniform. The uneven phase image reveals the surface non-uniformity in detail. The film was uneven with presence of crevices on the surface. The film had a rough texture. On the contrary, the 3D surface image of the film developed using TSE were more uniform and smooth (FIG. 4c), as compared to single-screw film (FIG. 4c).

The average roughness for ODFs prepared using twin-screw HME was 122.7 nm while for that of single screw extruded film, it was 1825 nm. In summary, the films prepared by twin-screw HME are smoother than that of single-screw melt extrusion.

### Raman Spectroscopy:

Raman spectroscopy is a spectroscopic technique commonly used in chemistry to provide a structural fingerprint by which molecules can be identified. It can identify a particular molecule/API based on the Raman shift. The formulated ODFs were analyzed using Raman spectra for identification of stability of active in the delivery system after processing using HME. The Raman spectra was recorded with a LabRam HR800 (Horiba Jovan Yvon, UK) equipped with a 534-nm Ar—Ne laser. The laser excitation was focused using a 50 objective (Olympus Corporation, Japan) and the scattered light was totally transmitted through the notch filter towards the confocal hole and entrance slit of the spectrograph. The stokes-shifted Raman scatter was dispersed using a 1800 groove/min grating onto a peltier-cooled changed-coupled device (CCD, Andor Technology PLC) to capture a spectrum. Each sample had their spectra measured using near infrared excitation (at 534 nm) of 250 mW and continuous readout of Raman shift spectrum from 0 to 3500 cm⁻¹. Spectra were acquired using a 50× objective and a 300 mm confocal hole. A 600 lines/mm rotatable diffraction grating along a path length of 800 mm was used to simultaneously scan a range of frequencies. Raman spectra were collected using a Synapse CCD detector (1024 pixels).

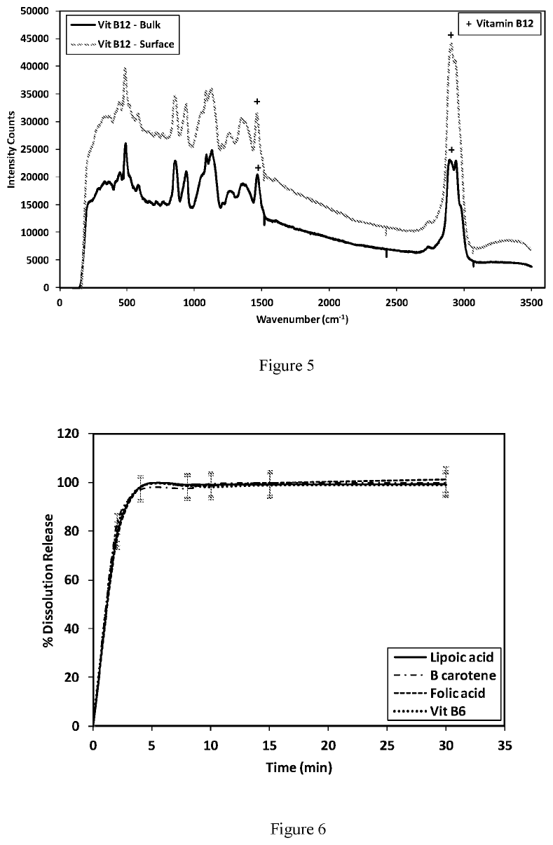

Raman spectra of the ODF containing Vitamin B12 (Example 6) are shown in FIG. 5. Vitamin B12 exhibit characteristics Raman shift at 1500 and 2985 cm⁻¹ (2). The Raman spectra of the ODF containing Vitamin B12 shows strong peaks at 1462.97 (+) and 2889.49 cm⁻¹ (+). This indicates that the drug does not degrade in the twin-screw HME after processing and remains stable in the final film formulation. In order to observe the uniformity of active/API distribution in the polymeric film, Raman spectra were recorded at surface and in bulk of the film sample. The strong Raman shifts of Vitamin B12 were observed in both of these scans. Hence, it can be concluded that films prepared using twin-screw HME exhibit uniform distribution and stable active.

### In Vitro Disintegration Time Study:

The disintegration time limit of 30 s or less for orally disintegrating tablets, described in CDER guidance can be applied for fast dissolving oral strips. Although, no official guidance is available for oral fast disintegrating films/strips, this may be used as a qualitative guideline for quality control test or at development stage. The in vitro disintegration time of the ODF formulations (20×20 mm) was determined using a disintegration tester (Electrolab, India) with phosphate buffer pH 6.8 at 37.0±0.5° C. The disintegration time was defined as the time taken for ODF to completely disintegrate with no solid residue remaining on the screen. A total of six ODF samples were run for each formulation.

The mean in vitro disintegration time was 45-50 s for all the ODFs developed. There was no statistically significant difference (p>0.05) in the in vitro disintegration time among the different ODFs formulations.

### In Vitro Dissolution Studies:

The in vitro drug dissolution study was carried out in 500 mL of phosphate buffer, pH 6.8 at 37.0±0.5° C. and using USP II paddle method at a stirring speed of 50 rpm. Each square cut film sample was added into the dissolution media and appropriate aliquots were withdrawn at specific time intervals for 30 min. The drug concentration is measured by a UV spectrophotometer at the λₘₐₓ of respective actives. Four active components of three ODF samples (Examples 3-5) were analyzed for each formulation.

FIGS. 6 and 17 shows the dissolution profiles of the developed formulations using twin-screw HME. It was observed that the mean dissolution for all the ODFs was 79% at 2 min. The mean dissolution reached 98.78% within 10 min. Hence, films developed using twin-screw HME were showing desirable performance with respect to dissolution of the actives.

A stock solution of 0.25 mg/ml of Ondansetron HCl were prepared in methanol and measured absorbance at 216 nm.

A stock solution of 0.5 mg/ml of diphenhydramine HCl were prepared in water and measured absorbance at 254 nm.

A stock solution of 1000 μg/ml of alpha lipoic acid were prepared in methanol and measured absorbance at 322 nm.

Standard β-carotene for identification was prepared in DCM to obtain 1000 μg/mL and measured absorbance at 461 nm.

2 ml of the standard and sample solution of folic acid was taken in marked test tubes. In each test tube, 2 ml of 0.02% potassium permanganate solution, 2 ml 2% sodium nitrate solution, 2 ml 4 M hydrochloric acid solution, 1 ml 5% ammonium sulphamate solution and 1 ml dye solution (0.1% N, N diethyl aniline dye solution in iso propyl alcohol) added and mixed well, then kept for 15 minute at room temperature. It was absorbance recorded at 535 nm against blank.

2 ml of the standard and sample solution of Vitamin B6 was taken in marked test tubes. In each test tube, 1 ml of ammonium buffer (in water), 1 ml of 20% sodium acetate (in water), 1 ml of 5% boric acid (in water) and 1 ml dye (2,6-di-chloroquinine chorimide) solution added and mixed well. It was absorbance recorded at 650 nm against blank.

TABLE 13
Assay of actives.

| | Wavelength | Solvent used for Stock | Result |
|---|---|---|---|
| Actives | (λmax in nm) | solution | (%) |
| Lipoic acid | 322 | Methanol | 100.5 |
| B carotene | 461 | Dicloromethane (DCM) | 98.28 |
| Folic acid | 535 | 3% dipotassium | 99.1 |
| | | phosphate | |
| Vit B6 | 650 | Water | 99.3 |

Table 13 illustrates the assays of the active mentioned above. The assay were prepared of standard and sample solutions of four actives, that is, lipoic acid, β-carotene, folic acid and Vitamin B6 respectively. The absorbance was measured at different wavelengths and it was found that the wavelengths matched for the standard and samples solutions for each actives.

### Example 6

### Analysis of Mouth Freshener Film (Example 1 in Table 7)

The mouth freshener film was evaluated on several parameters in order to check the quality of the finished film. These parameters and the evaluations results have been described below in detail.

### Folding Endurance:

The folding endurance of the films was determined by repeatedly folding one film at the same place till it broke or folded up to 300 times, which is considered satisfactory to reveal good film properties. The number of times film could be folded at the same place without breaking gave the value of the folding endurance. This gives the indication of the brittleness of the film.

F=log 10 d

F=folding endurance
d=number of double folds

### Mechanical Properties:

Mechanical properties of films were evaluated using a CT3 Brookfield texture analyzer equipment equipped with a 5 Kg load cell. films are held between two clamps positioned between 3 cm. During measurement the films were pulled at rate of 2 mm/sec. The force and elongation were measured when film breaks. Two mechanical properties namely tensile strength and % elongation were calculated.
a) Tensile strength:

Tensile strength=Load at failure×100/film thickness×film width
b) Percent Elongation

Elongation=Increase in length/Original length×100

### In Vitro Disintegration Test:

The disintegration time limit of 30 s or less for orally disintegrating tablets described in CDER guidance can be applied fast dissolving oral strips. Although, no official guidance is available for oral fast disintegrating films/strips, this may be used as a qualitative guideline for quality control test or at development stage. Pharmacopoeial disintegration test apparatus may be used for this study. For both methods only a small amount of medium is used, so natural conditions could be simulated. Due to the use of small amount of medium, the dissolved drug substance could not be measured by spectral analysis.

### Petri Dish Method

2 mL of phosphate 6.8 buffer was placed in a Petri dish and one film was added on the surface of the water and the time measured until the oral film was dissolved completely.

### Surface pH:

A combined pH electrode is used for this purpose. Fast dissolving film was slightly wetted with distilled water. The pH was measured by bringing the electrode in contact with the surface of the film and performed in triplicate.

### Contact Angle:

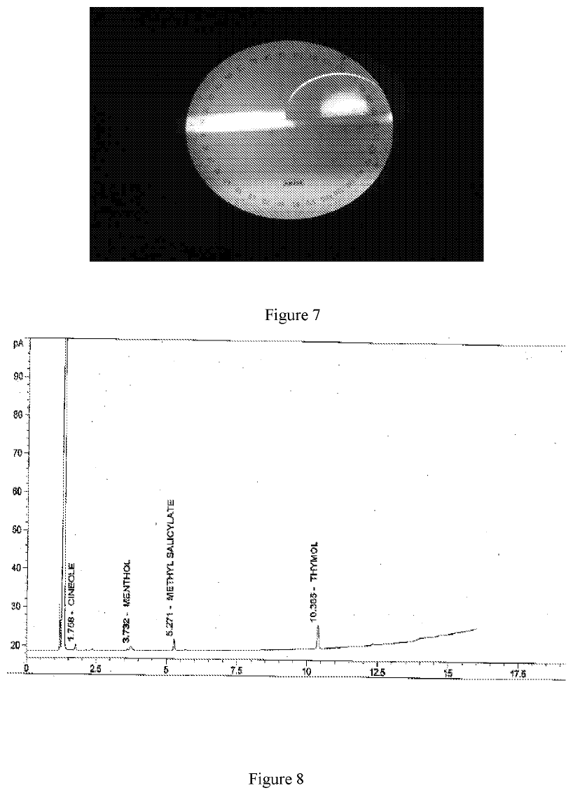

Contact angle measurement predicts the wetting behavior, disintegration time, and dissolution of oral film. These measurements are performed with help of goniometer and the measurements were done at room temperature. The water used to determine contact angle should be double distilled water. A drop of double distilled water is placed on the surface of dry film. Images of water droplet are recorded within 10 s of deposition by means of digital camera. Digital pictures can be analyzed by image J 1.28v software (NIH, USA) for angle determination. (FIG. 7)

TABLE 14
Evaluation results of mouth freshener film

| Tests | Observation | |
|---|---|---|
| Thickness | 0.16 | mm |
| Folding endurance | 1.19 | |
| Tensile strength | 5.29 | |
| % elongation | 10 | |
| In Vitro disintegration time | 10 | sec. |

| | |
|---|---|
| Surface pH | 5.95 |
| Contact angle | 22° |

As illustrated in Table 14, the film formulations demonstrated excellent D.T of 8-11 s, which was attributed to the thickness of the film and presence of water-soluble materials (contact angle—22°) in the film. The most crucial parameter for disintegration is the low thickness of the film. As the films had a thickness range of 0.14-0.16 mm, they aided in the faster disintegration of all film formulations. In addition to the low thickness of the film, the amount of saliva in the oral cavity is very critical for rapid disintegration. The normal flow of saliva in a healthy person is 0.34 mL/min, and it can be increased by the addition of agents that simulate salivary production. Citric acid is the most preferred saliva-stimulating agent, and it increases salivary flow. With the addition of citric acid, the pH of the films was found to be in the range of 5-6 and it could contribute in rapid disintegration of film product. Ideally, the film should have desirable mechanical properties so that it can remain intact during handling and transport. ODFs showed appropriate strength and % E. These excellent mechanical properties were attributed to the presence of sorbitol, citric acid, and active, which reduced film stiffness via disruption of intermolecular forces of the polymer owing to the accommodation of these compounds between the strands, thereby providing elasticity to the films.

### Gas Chromatography (GC) of Mouth Freshener Film

Gas Chromatography is quantitative study use to determine assay of actives. GC was done by Bangalore Testing Laboratories Pvt. Ltd., Bangalore (FIG. 8). The GC is used to observe whether the active is intact in the film without its decomposition. Table 15 illustrated all the characteristic peaks of the actives (menthol, thymol, methyl salicylate, eucalyptol) used in the composition. The results show that hot melt extrusion process does not decomposes the actives in the oral film composition as the characteristic peaks in GC for the actives were found to be prominent.

TABLE 15
Gas chromatography results of mouth freshener film

| Compound Name | Formulae (%) | GC Result (%) | Assay result (%) |
|---|---|---|---|
| Menthol content | 0.038 | 0.035 | 92.1 |
| Thymol | 0.093 | 0.097 | 104.3 |
| Methyl Salicylate | 0.056 | 0.052 | 92.8 |
| Eucalyptol | 0.034(cineole | 0.023(cineole | 96.6 |
| | content) | content) | |

### XRD of Menthol in Mouth Freshener Film:

Crystallinity in raw materials, hot-melt extrudates and physical mixtures was determined using A MiniFlex II desktop powder X-ray diffractometer (Rigaku Corporation, Japan) equipped with Ni-filtered, Cu Kβ radiation, at a voltage of 30 kV and a current of 15 mA. Samples were scanned in continuous mode within the angular range 3-40° 2θ with a scan speed of 2.0° min-1 and sample width of 0.03°.

XRD was used to investigate the physical state of the drug after HME process. Prominent peaks of active observed approximately at an angle—9, 18, 23°. The melt-extruded formulation did not show any peak, thus confirming the presence of drug in an amorphous form. The reasons behind the complete conversion of drug to an amorphous form were the high shear during extrusion and relatively high amounts of sorbitol. The presence of Menthol and excipients in an amorphous form aided the flexibility and clear appearance of the film as shown in FIG. 9. This illustrates that the hot melt extrusion process does not decomposes the active ingredient dispersed within the oral dispersible film composition.

### Thermal Analysis:

Thermogravimetric analysis (TGA) studies (Perkin Elmer Pyris 1, Shelton, Conn., USA) were performed to estimate the thermal stability of the actives and excipients during HME processing. Data were analyzed using Pyris software. The API excipients were heated from 30-360° C. at 20° C./min.

TGA is very critical before performing HME because the drug and excipients are exposed to high temperature during the extrusion process, and there are possibilities of drug degradation or thermally-induced chemical reactions or both. Results specified that Active, polymer, and excipients were chemically stable and all materials had excellent thermal stability and fit for the melt extrusion process.

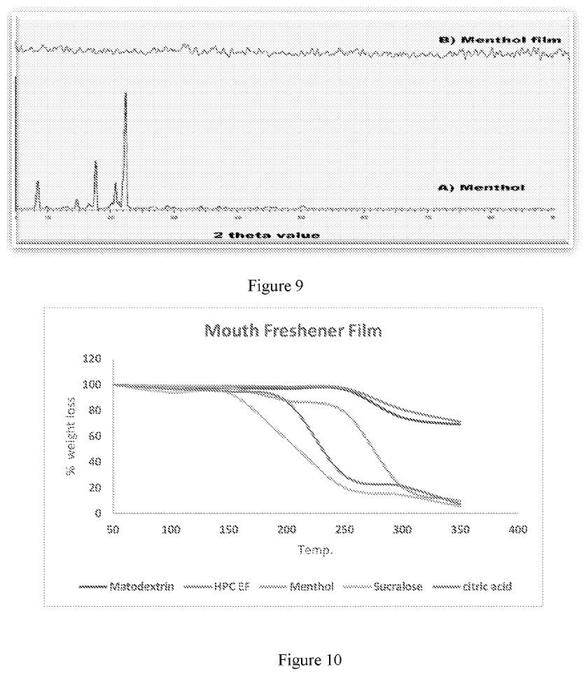

The TGA as showed in FIG. 10 illustrated that The TGA results specified that active, polymer, and excipients were chemically stable in the HME processing temperature range. Sucralose demonstrated a loss of weight (<7%), which was attributed to the moisture present in the excipient. These results confirmed that all materials had excellent thermal stability and fit for the melt extrusion process.

### High Performance Liquid Chromatography (HPLC) Evaluation of Ondansetron Film (Example 13 of Table 10)

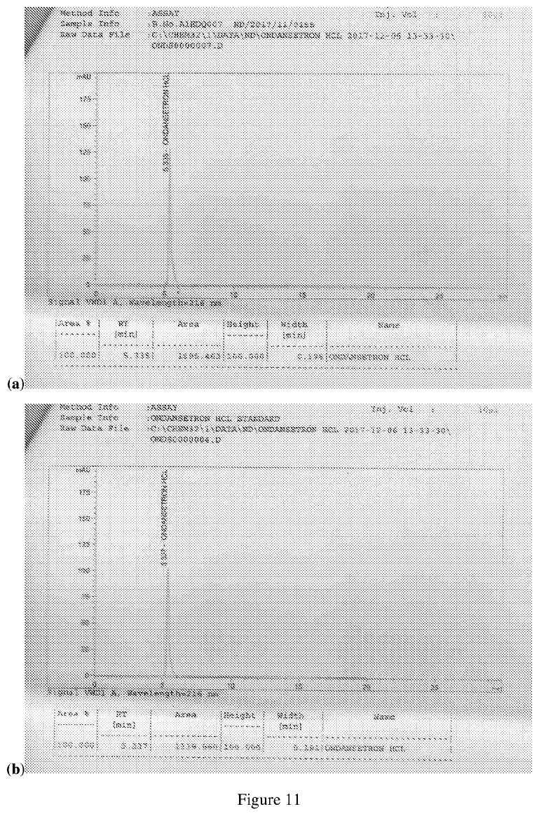

The oral dispersible film for Example 13 was subjected to HPLC analysis in order to check the presence and purity of the active in the film. The analysis was also conducted at Bangalore Testing Laboratories Pvt. Ltd., Bangalore. The results of the comparison between the assay sample of the film and the standard sample have been displayed in FIG. 11a 11b respectively. It was found that the active dispersed in Example 13 (ondansetron) showed a distinct peak at a retention time [RT (min)] of 5.335 in the assay of the dispersible film. The RT of the assay was similar to that of the standard ondansetron sample (RT=5.337 min). This showed that the hot melt extrusion process neither decomposes nor compromises the purity of the actives in the oral dispersible film composition.

### Characterization and Studies Involving Probiotic Film (Example 21 of Table 11) Strain Identification and Characterization

Lyophilized sample of lactobacilli acidophilus were given by Siddon Biotech, India as gift sample. The primary identification of the strain was based on gram staining and morphology. Molecular characterization by 16S rRNA partial gene sequencing was performed for strain identification.

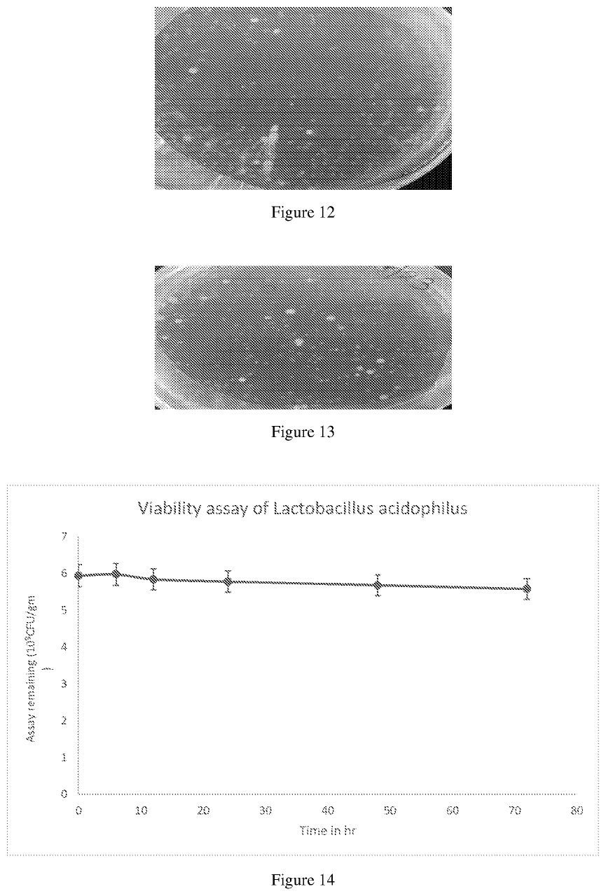

The method used for microbiological viability assay of Lactobacillus acidophilus were enumerated in the MRS medium. Serial dilutions of Lactobacillus acidophilus were prepared in normal saline. These cultures were plated by pour plate technique. The plates were incubated at temperature of 37° C. for 72 hours. The procedures were carried out in triplicate using aseptic techniques. The colony forming units (CFU) of bacteria were counted (see FIGS. 12 and 13). The cell viability studies (FIG. 14) indicate good stability of the strain.

### Excipient Compatibility Studies

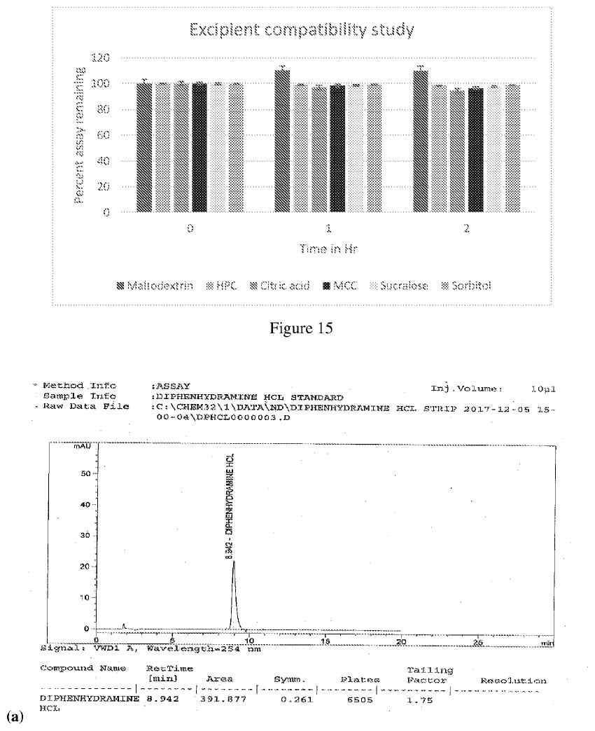

Binary blends of Lactobacillus acidophilus spores powder with excipients were prepared and stored in dark stability chamber maintained at temperature of 40° C.±2° C. and 75%±5% RH conditions. Samples were withdrawn after 2 hr and analyzed for microbiological assay (see FIG. 15).

No significant differences (p>0.05) in the number of viable bacteria were found between 0-48 hr in Probiotic oral dispersible film. However, maltodextrin and other excipient were found to be compatible and were able to maintain a therapeutic level of bacteria (10⁹ CFU/strip) during the total period of storage. Thus, indicating acceptable bio-compatibility.

### HPLC Assay of Films Containing Diphenhydramine Hydrochloride (Example 17 of Table 11)

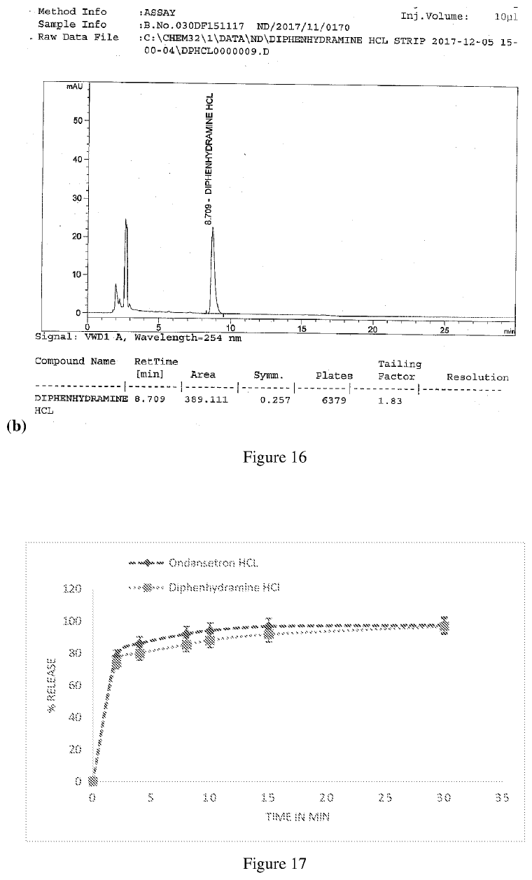

The oral dispersible film for Example 17 was subjected to HPLC analysis in order to check the presence and purity of the active in the film. The analysis was also conducted at Bangalore Testing Laboratories Pvt. Ltd., Bangalore. The results of the comparison between the assay sample of the film and the standard sample have been displayed in FIGS. 16a and 16b respectively. It was found that the active dispersed in Example 17 (diphenhydramine HCl) showed a distinct peak at a retention time [RT (min)] of 8.709 in the assay of the dispersible film. The RT of the assay was similar to that of the standard Diphenhydramine sample (RT=8.942 min). This showed that the hot melt extrusion process neither decomposes nor compromises the purity of the actives in the oral dispersible film composition.

### Ex Vivo Mucoadhesive Test for Canker Sore ODF (Examples 19 and 20 of Table 11)

The mucoadhesive strength of buccal film was determined on goat buccal mucosa (obtained from local market) by measuring the force of detachment or the force of adhesion. The goat buccal mucosa was fixed on the internal side of a beaker. The patch was wetted with 100 μL of phosphate buffer pH 6.8 and pasted to the rabbit buccal tissue by applying a light force with a fingertip for 1 min. The beaker was filled with 300 mL phosphate buffer pH 6.8 and kept at 37±1° C. Then, the beaker was magnetically stirred at 50 rpm stirring rate to simulate the buccal cavity environment. The time required for detachment of these patches from the buccal mucosa was recorded. The experiment was performed in triplicate and the results were analyzed for mean and SD.

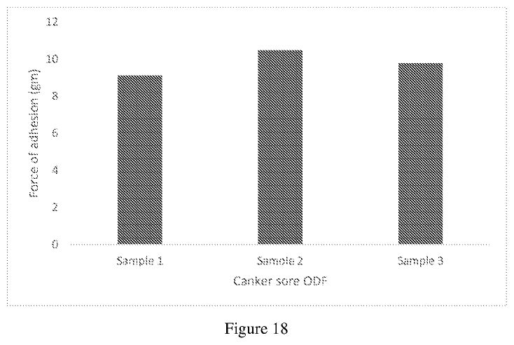

The mucoadhesion strength of formulations was 9.79±0.19 g as shown in FIG. 18. It was observed that the longest adhesion time and the convenient bioadhesion strength reflect the different behavior of maltodextrin, HPC, Sodium alginate and sodium CMC which, in addition to maltodextrin and HPC has film-forming polymers, possesses non-neglectable mucoadhesive properties that enhanced mucoadhesion. This study was sustained on the assumption that ionizable polymers exhibit the best mucoadhesive characteristics.

### ADVANTAGES GAINED IN THE EXAMPLE ILLUSTRATIVE PROCESS IN THIS SUBJECT MATTER

The present oral dispersible film composition was prepared using various components with specific roles. The weight percentage ranges of these components were optimized by conducting various experiments to obtain the film composition of desired attributes.

The oral dispersible film composition thus obtained was a fast dissolving film exhibiting an in-vitro release of at least 60% w/w in 1-2 minutes.

It was found that the film formed was uniform with even distribution/dispersion of the active therein. The film was found to show excellent tensile strength and folding endurance without the decomposition of the actives.

1. An oral dispersible film composition comprising:
a) maltodextrin; and
b) hydroxypropyl cellulose,
wherein the weight ratio of maltodextrin to hydroxypropyl cellulose is in the range of 1:1-3:1.

2. The oral dispersible film composition as claimed in claim 1, comprising at least one plasticizer selected from the group consisting of sorbitol, glycerol, polyethylene glycol (PEG), triethylcitrate (TEC), propylene glycol (PG), and combinations thereof.

3. The oral dispersible film composition as claimed in claim 1, wherein the weight ratio of maltodextrin to hydroxypropyl cellulose to plasticizer is in the range of 3:1:1-3:1:3.

4. The oral dispersible film composition as claimed in claim 1, wherein the oral dispersible film composition comprises at least one active ingredient dispersed in a matrix comprising the oral dispersible film composition.

5. The oral dispersible film composition as claimed in claim 4, wherein the at least one active ingredient is selected from the group consisting of mouth freshener, pharmaceutical agent, probiotics, dietary supplement, antioxidant, nutraceutical and combinations thereof.

6. The oral dispersible film composition as claimed in claim 1, wherein the oral dispersible film composition has an average thickness in the range of 0.04-0.8 mm.

7. The oral dispersible film composition as claimed in claim 1, wherein the oral dispersible film composition is a fast dissolving film exhibiting an in-vitro release of at least 60% w/w in 1-2 minutes.

8. An oral dispersible film composition comprising:
a) at least one active ingredient has a weight percentage in the range of 0.01-45% with respect to the composition and is selected from the group consisting of menthol, vitamin B6, folic acid, lipoic acid, β-carotene, vitamin B12, cocoa powder, calcium, vitamin D3, vitamin C, green tea, caffeine, ondansetron hydrochloride, memantine hydrochloride, isosorbide dinitrate, risperidone, diphenhydramine hydrochloride, ibuprofen, benzocaine, probiotics, brompheniramine maleate, chlorpheniramine maleate, carbinoxamine maleate, clemastine fumarate, dexchlorpheniramine maleate, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, loratadine, brompheniramine, dexbropheniramine, fexofenadine, cetirizine, famotidine, ranitidine, aspirin, acetaminophen, ketoprofen, diflunisal, fenoprofen, naproxen, tolmetin sodium, indomethacin, flurbiprofen sodium, celecoxib, valdecoxib, rofecoxib, sildenafil citrate, tadalafil, catechu, and combinations thereof.
b) maltodextrin has a weight percentage in the range of 28-65% with respect to the composition.
c) hydroxypropyl cellulose has a weight percentage in the range of 10-30% with respect to the composition.
d) at least one plasticizer has a weight percentage in the range of 1-15% with respect to the composition,
wherein the weight ratio of maltodextrin to hydroxypropyl cellulose to plasticizer is in the range of 3:1:1-3:1:3.

9. The oral dispersible film composition as claimed in claim 1, optionally comprising at least one saliva stimulating agent selected from the group consisting of citric acid, sodium croscarmellose, tartaric acid, lactic acid, ascorbic acid, malic acid, and combinations thereof, and having a weight percentage in the range of 0.1-5% with respect to the composition.

10. The oral dispersible film composition as claimed in claim 1, optionally comprising at least one pharmaceutically acceptable excipient selected from the group consisting of anti-sticking agent, sweetener, preservative, lubricant, flavoring agent, mucoadhesive agent, and combinations thereof.

11. The oral dispersible film composition as claimed in claim 10, wherein the anti-sticking agent has a weight percentage in the range of 0.1-8.0% with respect to the composition and is selected from the group consisting of microcrystalline cellulose, sodium lauryl sulfate, silicon dioxide, tween, polyoxyethylene alkylethers, bezthonium chloride, dibutyl tartrate, and combinations thereof; the sweetener has a weight percentage in the range of 0.1-5.0% with respect to the composition and is selected from the group consisting of sucralose, xylose, ribose, glucose, mannose, galactose, fructose, dextrose, sucrose, maltose, partially hydrolyzed starch, corn syrup solids, xylitol, mannitol, saccharin salts, cyclamate salts, acesulfam-K, aspartame, neotame, and combination thereof; the preservative has a weight percentage in the range of 0.05-3.0% with respect to the composition and is selected from the group consisting of sodium benzoate, benzalkonium chloride, potassium sorbate, methyl paraben, propyl paraben, and combinations thereof; the lubricant has a weight percentage in the range of 0.1-1% with respect to the composition and is selected from the group consisting of sodium hyaluronate, sucrose esters, glyceryl behenate (stelliesters), stearic acid, dimethyl phthalate, diethyl phthalate, dibutyl phthalate, tributyl citrate, triethyl citrate, acetyl citrate, triacetin, dioctyl adipate, diethyl adipate, di(2-methylethyl) adipate, dihexyl adipate, partial fatty acid esters of sugars, polyethylene glycol fatty acid esters, polyethylene glycol fatty alcohol ethers, polyethylene glycol sorbitan fatty acid esters, 2-ethoxy ethanol, ethyl alcohol, propyl alcohol, butyl alcohol, pentyl alcohol, hexyl alcohol, heptyl alcohol, octyl alcohol, dibutyl tartrate, castor oil, and combinations thereof; the flavoring agent has a weight percentage in the range of 0.5-6% with respect to the composition and is selected from the group consisting of orange flavor, coffee, green mango flavor, cocoa flavor, banana flavor, strawberry flavor, custard apple flavor, watermelon flavor, pineapple flavor and various fruit flavors, mint flavor, peppermint flavor, and combinations thereof; and the mucoadhesive agent has a weight percentage in the range of 0.1-8.0% and is selected from the group consisting of sodium alginate, sodium carboxymethyl cellulose, and combinations thereof.

12. A process for preparation of the oral dispersible film composition as claimed in claim 1, comprising the steps of:
a) contacting maltodextrin, hydroxypropyl cellulose, plasticizer, at least one active ingredient, at least one saliva stimulating agent, and excipient to obtain a first mixture.
b) extruding the first mixture to obtain the oral dispersible film composition.

13. The process as claimed claim 12, comprising the steps of:
a) contacting at least one active ingredient, maltodextrin, hydroxypropyl cellulose, at least one saliva stimulating agent, sweetener, and flavoring agent to obtain a first mixture;
b) contacting the first mixture, plasticizer, and anti-sticking agent to obtain a second mixture; and
c) extruding the second mixture to obtain the oral dispersible film composition,
wherein the weight ratio of maltodextrin to hydroxypropyl cellulose is in the range of 1:1-3:1.

14. The process for preparation of the oral dispersible film composition as claimed in claim 13, wherein extruding the second mixture is carried out through a twin screw hot melt extrusion method.

15. The process for preparation of the oral dispersible film composition as claimed in claim 13, wherein extruding the second mixture is carried out at a temperature in the range of 80-110° C. to obtain the oral dispersible film composition.

16. A system for extruding the second mixture as claimed in claim 13, comprising:
a) a hopper zone for feeding the second mixture as claimed in claim 13;
b) a conveying zone for transporting, processing and homogenizing the second mixture;
c) an orifice zone for shaping the homogenized second mixture to obtain a film of the second mixture;
d) a downstream ancillary equipment zone for collecting the film of the oral dispersible film composition, wherein the oral dispersible film composition is in the form of a film having an average thickness in the range of 0.04-0.8 mm.

17. The process for preparation of the oral dispersible film composition as claimed in claim 13, wherein the process of extruding the second mixture comprises the steps of:
a) feeding the second mixture in the hopper zone;
b) processing the second mixture to obtain the oral dispersible film composition in the conveying zone;
c) shaping the oral dispersible film composition obtained from the conveying zone into a film in the orifice zone; and
d) cooling and collecting the film of the oral dispersible film composition from the downstream ancillary equipment zone.

18. The process for preparation of the oral dispersible film composition as claimed in claim 16, wherein the process of extruding the second mixture comprises the steps of:
a) feeding the second mixture in the hopper zone;
b) processing the second mixture to obtain the oral dispersible film composition in the conveying zone;
c) shaping the oral dispersible film composition obtained from the conveying zone into a film in the orifice zone; and
d) cooling and collecting the film of the oral dispersible film composition from the downstream ancillary equipment zone.
